(19) United States
(12) Patent Application Publication
(10) Pub. No.: US 2024/0368235 A1
(43) Pub. Date: Nov. 7, 2024

(54) Disease Treatment Drug Based on Mesenchymal-Stem-Cell Mobilization

(71) Applicants: StemRIM Inc., Osaka (JP); OSAKA UNIVERSITY, Osaka (JP)

(72) Inventors: Katsuto TAMAI, Osaka (JP); Takashi SHIMBO, Osaka (JP); Takehiko YAMAZAKI, Osaka (JP)

(21) Appl. No.: 18/776,908

(22) Filed: Jul. 18, 2024

Related U.S. Application Data
(62) Division of application No. 17/282,872, filed on Apr. 5, 2021.

(30) Foreign Application Priority Data
Oct. 5, 2018 (JP) 2018190089

(51) Int. Cl. C07K 14/47; A61K 38/00; A61P 1/00; A61P 17/06; A61P 37/06

(52) U.S. Cl. CPC C07K 14/4713; A61P 1/00; A61P 17/06; A61P 37/06; A61K 38/00; C07K 2319/09

(57) ABSTRACT

The present inventors identified many nuclear proteins contained in the extract of skin tissue by mass spectrometry, randomly selected multiple partial amino acid sequences of the nuclear proteins, chemically synthesized peptides consisting of the partial amino acid sequences, and examined their activity of mobilizing mesenchymal stem cells. As a result, it was found that these multiple peptides show the activity of mobilizing mesenchymal stem cells into peripheral blood, even though their amino acid sequences are completely different from each other. The inventors also found that fragment peptides of the nuclear proteins have therapeutic effects on diseases characterized by inflammation and abnormalities of the immune system (e.g., inflammatory bowel disease and psoriasis). Based on these findings, a new regenerative medicine technology that can overcome the problems of cell transplantation therapy is provided.

### CROSS REFERENCE TO RELATED APPLICATIONS

This application is a division of U.S. patent application Ser. No. 17/282,872, filed Apr. 5, 2021; which is a National Stage Application of International Application No. PCT/JP2019/039231, filed Oct. 4, 2019; which claims priority to Japanese Application No. 2018-190089, filed Oct. 5, 2018.

### SEQUENCE LISTING

The Sequence Listing for this application is labeled “SPO-154D1-SeqList-18Jul24.xml”, which was created on Jul. 18, 2024, and is 161 KB. The entire content is incorporated herein by reference in its entirety.

### TECHNICAL FIELD

The present application relates to compositions for mobilizing mesenchymal stem cells and agents for treating diseases based on the mobilization of mesenchymal stem cells.

### BACKGROUND ART

Mesenchymal stem cells contained in bone marrow fluid and the like have the ability to differentiate into various tissues (pluripotency) such as bone, cartilage, fat, muscle, nerve, and epithelium. In recent years, attempts have been widely made to perform regenerative medicine (cell transplantation therapy) using bone marrow-derived mesenchymal stem cells proliferated by culture. However, collection of bone marrow blood containing mesenchymal stem cells is done with an invasive technique which inserts a thick needle into the iliac bone repeatedly, thereby placing a large burden on the donor. In addition, mesenchymal stem cells gradually lose their proliferative ability and pluripotency when subcultured continuously in vitro. Furthermore, culturing mesenchymal stem cells based on high quality control that guarantees the safety of in vivo transplantation requires special culture equipment such as CPC (cell processing center), so the current situation is that it can be carried out only at a limited number of universities and companies.

### CITATION LIST

### Non-Patent Literature

[NPL 1] PNAS 2011 Apr. 19; 108 (16): 6609-14

### PATENT LITERATURE

[PTL 1] WO2008/053892
[PTL 2] WO2009/133939
[PTL 3] WO2012/147470

### SUMMARY OF INVENTION

### Technical Problem

An objective of the present application is to develop a new regenerative medicine technology that can overcome the problems of cell transplantation therapy.

### Solution to Problem

The present inventors identified a large number of nucleoproteins contained in an extract of skin tissue by mass spectrometry, randomly selected a plurality of partial amino acid sequences of the identified nucleoproteins, and chemically synthesized peptides consisting of the partial amino acid sequences. Then, their activity of mobilizing mesenchymal stem cells was examined. As a result, it was found that these peptides show an activity of mobilizing mesenchymal stem cells into peripheral blood, even though their amino acid sequences are completely different from each other. It was also found that fragment peptides of the nucleoproteins have therapeutic effects on diseases characterized by inflammation and abnormalities of the immune system (e.g., inflammatory bowel disease and psoriasis). Based on these findings, a new regenerative medicine technology to overcome the problems of cell transplantation therapy is provided.

Specifically, the present application provides the following:

[1]

A composition for use in mobilizing mesenchymal stem cells to peripheral blood, which comprises a nuclear protein or a fragment peptide thereof.

[2]

A composition for use in treatment of a disease or pathological condition in a subject by mobilizing mesenchymal stem cells to peripheral blood, which comprises a nuclear protein or a fragment peptide thereof.

[3]

The composition of [2], wherein the treatment of a disease or pathological condition is selected from inflammation-suppressing therapy, immunomodulatory therapy, tissue regeneration-inducing therapy, and tissue fibrosis-suppressing therapy.

[4]

The composition of [2], wherein the disease or pathological condition is selected from an inflammatory disease, an autoimmune disease, a disease accompanied by tissue damage, ischemia, or necrosis, and a fibrotic disease.

[5]

The composition of [2], wherein the disease or pathological condition is selected from inflammatory bowel disease and psoriasis.

[6]

A composition for use in treatment of a disease selected from inflammatory bowel disease and psoriasis, which comprises a nuclear protein or a fragment peptide thereof.

[7]

The composition of any one of [1] to [6], wherein the nuclear protein or fragment peptide thereof comprises a nuclear localization signal.

[8]

The composition of any one of [1] to [7], wherein the nuclear protein or fragment peptide thereof is a nuclear protein involved in transcription regulation or a fragment peptide thereof.

[9]

The composition of any one of [1] to [8], wherein the nuclear protein or fragment peptide thereof is a nuclear protein selected from the following or a fragment peptide thereof:
(1) BTF3 protein;
(2) SUPT16H protein;
(3) YBX1 protein;
(4) NPM1 protein;
(5) PA2G4 protein;
(6) PFDN5 protein;
(7) PSMC3 protein;
(8) HNRNPK protein; and
(9) a nuclear protein functionally equivalent to a protein selected from (1) to (8).
   [10]

The composition of any one of [1] to [9], wherein the nuclear protein or fragment peptide thereof is a nuclear protein selected from the following or a fragment peptide thereof:
(a) a nuclear protein comprising an amino acid sequence selected from SEQ ID NOs: 1 to 34;
(b) a nuclear protein comprising an amino acid sequence resulting from substitution, deletion, insertion, or addition of one or more amino acids in an amino acid sequence selected from SEQ ID NOs: 1 to 34; and
(c) a nuclear protein comprising an amino acid sequence with a sequence identity of about 80% or higher with an amino acid sequence selected from SEQ ID NOs: 1 to 34.
   [11]

The composition of any one of [1] to [10], wherein the fragment peptide of the nuclear protein is a fragment peptide selected from the following:
(a) a nuclear protein fragment peptide consisting of a portion of an amino acid sequence selected from SEQ ID NOs: 1 to 34;
(b) a nuclear protein fragment peptide comprising an amino acid sequence selected from SEQ ID NOs: 35 to 56;
(c) a nuclear protein fragment peptide consisting of a portion of an amino acid sequence selected from SEQ ID NOs: 35 to 56;
(d) a nuclear protein fragment peptide comprising an amino acid sequence resulting from substitution, deletion, insertion, or addition of one or more amino acids in an amino acid sequence selected from SEQ ID NOs: 35 to 56; and
(e) a nuclear protein fragment peptide comprising an amino acid sequence with a sequence identity of about 80% or higher with an amino acid sequence selected from SEQ ID NOs: 35 to 56.
   [12]

A fragment peptide of a nuclear protein selected from the following:
(1) BTF3 protein;
(2) SUPT16H protein;
(3) YBX1 protein;
(4) NPM1 protein;
(5) PA2G4 protein;
(6) PFDN5 protein;
(7) PSMC3 protein;
(8) HNRNPK protein; and
(9) a nuclear protein functionally equivalent to a protein selected from (1) to (8).
   [13]

The fragment peptide of [12], comprising a nuclear localization signal.

[14]

The fragment peptide of [12] or [13], which is a fragment peptide of a nuclear protein selected from the following:
(a) a nuclear protein comprising an amino acid sequence selected from SEQ ID NOs: 1 to 34;
(b) a nuclear protein comprising an amino acid sequence resulting from substitution, deletion, insertion, or addition of one or more amino acids in an amino acid sequence selected from SEQ ID NOs: 1 to 34; and
(c) a nuclear protein comprising an amino acid sequence with a sequence identity of about 80% or higher with an amino acid sequence selected from SEQ ID NOs: 1 to 34.
   [15]

The fragment peptide of any one of [12] to [14], which is selected from the following:
(a) a nuclear protein fragment peptide consisting of a portion of an amino acid sequence selected from SEQ ID NOs: 1 to 34;
(b) a nuclear protein fragment peptide comprising an amino acid sequence selected from SEQ ID NOs: 35 to 56;
(c) a nuclear protein fragment peptide consisting of a portion of an amino acid sequence selected from SEQ ID NOs: 35 to 56;
(d) a nuclear protein fragment peptide comprising an amino acid sequence resulting from substitution, deletion, insertion, or addition of one or more amino acids in an amino acid sequence selected from SEQ ID NOs: 35 to 56; and
(e) a nuclear protein fragment peptide comprising an amino acid sequence with a sequence identity of about 80% or higher with an amino acid sequence selected from SEQ ID NOs: 35 to 56.

### [A1]

A method for mobilizing mesenchymal stem cells to peripheral blood, comprising administering to a subject an effective amount of a nuclear protein or a fragment peptide thereof.

### [A2]

A method for treating a disease or pathological condition in a subject by mobilizing mesenchymal stem cells to peripheral blood, which comprises administering to the subject an effective amount of a nuclear protein or a fragment peptide thereof.

### [A3]

The method of [A2], wherein the treatment of a disease or pathological condition is selected from inflammation-suppressing therapy, immunomodulatory therapy, tissue regeneration-inducing therapy, and tissue fibrosis-suppressing therapy.

### [A4]

The method of [A2], wherein the disease or pathological condition is selected from an inflammatory disease, an autoimmune disease, a disease accompanied by tissue damage, ischemia, or necrosis, and a fibrotic disease.

### [A5]

The method of [A2], wherein the disease or pathological condition is selected from inflammatory bowel disease and psoriasis.

### [A6]

A method for treating a disease selected from inflammatory bowel disease or psoriasis in a subject, which comprises administering to the subject an effective amount of a nuclear protein or a fragment peptide thereof.

### [A7]

The method of any one of [A1] to [A6], wherein the nuclear protein or fragment peptide thereof comprises a nuclear localization signal.

### [A8]

The method of any one of [A1] to [A7], wherein the nuclear protein or fragment peptide thereof is a nuclear protein involved in transcription regulation or a fragment peptide thereof.

### [A9]

The method of any one of [A1] to [A8], wherein the nuclear protein or fragment peptide thereof is a nuclear protein selected from the following or a fragment peptide thereof:
(1) BTF3 protein;
(2) SUPT16H protein;
(3) YBX1 protein;
(4) NPM1 protein;
(5) PA2G4 protein;
(6) PFDN5 protein;
(7) PSMC3 protein;
(8) HNRNPK protein; and
(9) a nuclear protein functionally equivalent to a protein selected from (1) to (8).

### [A10]

The method of any one of [A1] to [A9], wherein the nuclear protein or fragment peptide thereof is a nuclear protein selected from the following or a fragment peptide thereof:
(a) a nuclear protein comprising an amino acid sequence selected from SEQ ID NOs: 1 to 34;
(b) a nuclear protein comprising an amino acid sequence resulting from substitution, deletion, insertion, or addition of one or more amino acids in an amino acid sequence selected from SEQ ID NOs: 1 to 34; and
(c) a nuclear protein comprising an amino acid sequence with a sequence identity of about 80% or higher with an amino acid sequence selected from SEQ ID NOs: 1 to 34.

### [A11]

The method of any one of [A1] to [A10], wherein the fragment peptide of the nuclear protein is a fragment peptide selected from the following:
(a) a nuclear protein fragment peptide consisting of a portion of an amino acid sequence selected from SEQ ID NOs: 1 to 34;
(b) a nuclear protein fragment peptide comprising an amino acid sequence selected from SEQ ID NOs: 35 to 56;
(c) a nuclear protein fragment peptide consisting of a portion of an amino acid sequence selected from SEQ ID NOs: 35 to 56;
(d) a nuclear protein fragment peptide comprising an amino acid sequence resulting from substitution, deletion, insertion, or addition of one or more amino acids in an amino acid sequence selected from SEQ ID NOs: 35 to 56; and
(e) a nuclear protein fragment peptide comprising an amino acid sequence with a sequence identity of about 80% or higher with an amino acid sequence selected from SEQ ID NOs: 35 to 56.

### [B1]

A nuclear protein or a fragment peptide thereof for use in mobilizing mesenchymal stem cells to peripheral blood.

### [B2]

A nuclear protein or a fragment peptide thereof for use in treatment of a disease or pathological condition in a subject by mobilizing mesenchymal stem cells to peripheral blood.

### [B3]

The nuclear protein or fragment peptide thereof of [B2], wherein the treatment of a disease or pathological condition is selected from inflammation-suppressing therapy, immunomodulatory therapy, tissue regeneration-inducing therapy, and tissue fibrosis-suppressing therapy.

### [B4]

The nuclear protein or fragment peptide thereof of [B2], wherein the disease or pathological condition is selected from an inflammatory disease, an autoimmune disease, a disease accompanied by tissue damage, ischemia, or necrosis, and a fibrotic disease.

### [B5]

The nuclear protein or fragment peptide thereof of [B2], wherein the disease or pathological condition is selected from inflammatory bowel disease and psoriasis.

### [B6]

A nuclear protein or a fragment peptide thereof for use in treatment of a disease selected from inflammatory bowel disease and psoriasis.

### [B7]

The nuclear protein or fragment peptide thereof of any one of [B1] to [B6], which comprises a nuclear localization signal.

### [B8]

The nuclear protein or fragment peptide thereof of any one of [B1] to [B7], which is a nuclear protein involved in transcription regulation or a fragment peptide thereof.

### [B9]

The nuclear protein or fragment peptide thereof of any one of [B1] to [B8], which is selected from the following:
(1) BTF3 protein;
(2) SUPT16H protein;
(3) YBX1 protein;
(4) NPM1 protein;
(5) PA2G4 protein;
(6) PFDN5 protein;
(7) PSMC3 protein;
(8) HNRNPK protein; and
(9) a nuclear protein functionally equivalent to a protein selected from (1) to (8).

### [B10]

The nuclear protein or fragment peptide thereof of any one of [B1] to [B9], which is selected from the following:
(a) a nuclear protein comprising an amino acid sequence selected from SEQ ID NOs: 1 to 34;
(b) a nuclear protein comprising an amino acid sequence resulting from substitution, deletion, insertion, or addition of one or more amino acids in an amino acid sequence selected from SEQ ID NOs: 1 to 34; and
(c) a nuclear protein comprising an amino acid sequence with a sequence identity of about 80% or higher with an amino acid sequence selected from SEQ ID NOs: 1 to 34.

### [B11]

The nuclear protein or fragment peptide thereof of any one of [B1] to [B10], which is selected from the following:
(a) a nuclear protein fragment peptide consisting of a portion of an amino acid sequence selected from SEQ ID NOs: 1 to 34;
(b) a nuclear protein fragment peptide comprising an amino acid sequence selected from SEQ ID NOs: 35 to 56;
(c) a nuclear protein fragment peptide consisting of a portion of an amino acid sequence selected from SEQ ID NOs: 35 to 56;
(d) a nuclear protein fragment peptide comprising an amino acid sequence resulting from substitution, deletion, insertion, or addition of one or more amino acids in an amino acid sequence selected from SEQ ID NOs: 35 to 56; and
(e) a nuclear protein fragment peptide comprising an amino acid sequence with a sequence identity of about 80% or higher with an amino acid sequence selected from SEQ ID NOs: 35 to 56.

### [C1]

Use of a nuclear protein or a fragment peptide thereof in manufacture of a medicament or reagent for mobilizing mesenchymal stem cells to peripheral blood.

### [C2]

Use of a nuclear protein or a fragment peptide thereof in manufacture of a medicament for treating a disease or pathological condition in a subject by mobilizing mesenchymal stem cells to peripheral blood.

### [C3]

The use of [C2], wherein the treatment of a disease or pathological condition is selected from inflammation-suppressing therapy, immunomodulatory therapy, tissue regeneration-inducing therapy, and tissue fibrosis-suppressing therapy.

### [C4]

The use of [C2], wherein the disease or pathological condition is selected from an inflammatory disease, an autoimmune disease, a disease accompanied by tissue damage, ischemia, or necrosis, and a fibrotic disease.

### [C5]

The use of [C2], wherein the disease or pathological condition is selected from inflammatory bowel disease and psoriasis.

### [C6]

Use of a nuclear protein or a fragment peptide thereof in manufacture of a medicament for treating a disease selected from inflammatory bowel disease and psoriasis.

### [C7]

The use of any one of [C1] to [C6], wherein the nuclear protein or fragment peptide thereof comprises a nuclear localization signal.

### [C8]

The use of any one of [C1] to [C7], wherein the nuclear protein or fragment peptide thereof is a nuclear protein involved in transcription regulation or a fragment peptide thereof.

### [C9]

The use of any one of [C1] to [C8], wherein the nuclear protein or fragment peptide thereof is a nuclear protein selected from the following or a fragment peptide thereof:
(1) BTF3 protein;
(2) SUPT16H protein;
(3) YBX1 protein;
(4) NPM1 protein;
(5) PA2G4 protein;
(6) PFDN5 protein;
(7) PSMC3 protein;
(8) HNRNPK protein; and
(9) a nuclear protein functionally equivalent to a protein selected from (1) to (8).

### [C10]

The use of any one of [C1] to [C9], wherein the nuclear protein or fragment peptide thereof is a nuclear protein selected from the following or a fragment peptide thereof:
(a) a nuclear protein comprising an amino acid sequence selected from SEQ ID NOs: 1 to 34;
(b) a nuclear protein comprising an amino acid sequence resulting from substitution, deletion, insertion, or addition of one or more amino acids in an amino acid sequence selected from SEQ ID NOs: 1 to 34; and
(c) a nuclear protein comprising an amino acid sequence with a sequence identity of about 80% or higher with an amino acid sequence selected from SEQ ID NOs: 1 to 34.

### [C11]

The use of any one of [C1] to [C10], wherein the fragment peptide of the nuclear protein is a fragment peptide selected from the following:
(a) a nuclear protein fragment peptide consisting of a portion of an amino acid sequence selected from SEQ ID NOs: 1 to 34;
(b) a nuclear protein fragment peptide comprising an amino acid sequence selected from SEQ ID NOs: 35 to 56;
(c) a nuclear protein fragment peptide consisting of a portion of an amino acid sequence selected from SEQ ID NOs: 35 to 56;
(d) a nuclear protein fragment peptide comprising an amino acid sequence resulting from substitution, deletion, insertion, or addition of one or more amino acids in an amino acid sequence selected from SEQ ID NOs: 35 to 56; and
(e) a nuclear protein fragment peptide comprising an amino acid sequence with a sequence identity of about 80% or higher with an amino acid sequence selected from SEQ ID NOs: 35 to 56.

### BRIEF DESCRIPTION OF DRAWINGS

FIG. 1 is a plot of the number of colonies obtained by culturing peripheral blood 14 hours after administration of saline or the peptides. The number of colonies is shown as a value converted per 1 mL of collected peripheral blood. The long horizontal bar represents the mean value, and the short horizontal bars represent the standard deviation.

FIG. 2 is a plot of the number of colonies obtained by culturing peripheral blood 14 hours after administration of saline or the peptides. The number of colonies is shown as a value converted per 1 mL of collected peripheral blood. The long horizontal bar represents the mean value, and the short horizontal bars represent the standard deviation.

FIG. 3 is a plot of the number of colonies obtained by culturing peripheral blood 16 hours after administration of saline or the peptides. The number of colonies is shown as a value per peripheral blood volume (about 800 μL) collected from one mouse. The long horizontal bar represents the average value, and the short horizontal bars represent the standard deviation.

FIG. 4 is a plot of the number of colonies obtained by culturing peripheral blood 24 hours after administration of saline or the peptides. The number of colonies is shown as a value per peripheral blood volume (about 800 μL) collected from one mouse. The long horizontal bar represents the average value, and the short horizontal bars represent the standard deviation.

FIG. 5 is a graph that shows the body weight change of the mice. In the graph, “saline” indicates the control group, and “NP-1” indicates the peptide NP-1 administration group. On the horizontal axis, the number of days indicates the number of days after the start of drinking the aqueous solution of dextran sulfate sodium (DSS), and triangle indicates the date of administration of physiological saline (control group) or the peptide (NP-1 administration group).

FIG. 6 is a graph that shows the body weight change of the mice. In the graph, “saline” indicates the control group, and “NP-2” indicates the peptide NP-2 administration group. On the horizontal axis, the number of days indicates the number of days after the start of drinking the aqueous solution of dextran sulfate sodium (DSS), and triangle indicates the date of administration of physiological saline (control group) or the peptide (NP-2 administration group).

FIG. 7 is a graph that shows the body weight change of the mice. In the graph, “saline” indicates the control group, and “NP-3” indicates the peptide NP-3 administration group. On the horizontal axis, the number of days indicates the number of days after the start of drinking the aqueous solution of dextran sulfate sodium (DSS), and triangle indicates the date of administration of physiological saline (control group) or the peptide (NP-3 administration group).

FIG. 8 is a graph that shows the body weight change of the mice. In the graph, “saline” indicates the control group, and “NP-4” indicates the peptide NP-4 administration group. On the horizontal axis, the number of days indicates the number of days after the start of drinking the aqueous solution of dextran sulfate sodium (DSS), and triangle indicates the date of administration of physiological saline (control group) or the peptide (NP-4 administration group).

FIG. 9 is a graph showing changes in auricular thickness of mice. “Non treat” indicates normal mice, “IMQ/saline” indicates the control group, and “IMQ/NP-3” indicates the peptide NP-3 administration group. The horizontal axis indicates the number of days after the start of imiquimod application, and the vertical axis (Δthickness) shows difference (An−A0) between the auricular thickness before the start of imiquimod application (Day 0) (A0) and the auricular thickness at each day after the start of imiquimod application (An; n=1-7).

### DESCRIPTION OF EMBODIMENTS

The present inventors previously found a substance having an activity of activating stem cells in a living body or mobilizing them to an injured tissue via peripheral circulation, and believe that the substance is promising as a new type of medicine that can overcome the weaknesses of cell therapy. Specifically, the present inventors have found that High Mobility Group Box 1 (HMGB1), which is a nuclear protein released from necrotic tissue, mobilizes cells that are positive for platelet-derived growth factor receptor α (PDGFRα), which play a role in inducing tissue regeneration in vivo (believed to be mesenchymal stem cells (MSC)), to a necrotic tissue through peripheral circulation, thereby suppresses inflammation of the necrotic tissue and promotes tissue regeneration. Further, the present inventors have also found that fragment peptides of HMGB1 exhibit an activity of mobilizing mesenchymal stem cells into peripheral blood and a tissue regeneration-inducing activity. Therefore, the activities shown by the HMGB1 fragment peptides do not appear to depend on three-dimensional structure.

HMGB1 is physiologically absent in the blood and is released into the blood by necrotic cells only when necrotic damage occurs. This suggests that mesenchymal stem cells recognize the presence of necrotic tissue in vivo by the exposure to HMGB1 or a fragment thereof, and are then mobilized into peripheral blood to suppress inflammation of the necrotic tissue and promote regeneration.

Here, the present inventors considered that nuclear proteins other than HMGB1 would also be released into the blood by necrotic damage proteins, and that nuclear proteins other than HMGB1 or fragments thereof may have similar mesenchymal stem cell-mobilizing activity as the HMGB1 protein or fragment peptides thereof. That is, the inventors considered that it would be possible to provide the theory that “mesenchymal stem cells recognize nuclear proteins or fragment peptides thereof that are not physiologically present in the blood and are mobilized to the blood”.

To prove this theory, the present inventors identified many nuclear proteins contained in skin tissue extracts by mass spectrometry, randomly selected multiple partial amino acid sequences of the nuclear proteins identified, chemically synthesized peptides consisting of the partial amino acid sequences, and examined them for the mesenchymal stem cell-mobilizing activity. As a result, the above theory was confirmed to be correct, as these multiple peptides showed activities to mobilize mesenchymal stem cells into peripheral blood even though their amino acid sequences were completely different from each other.

Further, the present inventors found that fragment peptides of the above-mentioned nuclear proteins show therapeutic effects against diseases characterized by inflammation and abnormalities of the immune system (such as inflammatory bowel disease and psoriasis). Specifically, it was confirmed that the fragment peptides of nuclear proteins suppress weight loss in a mouse model of inflammatory bowel disease and suppress skin thickening in a psoriasis model.

It is well known to those skilled in the art that mesenchymal stem cells exert anti-inflammatory, immunomodulatory, and antifibrotic effects. Further, it is also well known to those skilled in the art that mesenchymal stem cells can exert a regeneration-promoting action on damaged tissues, as they have multipotency in that they can differentiate into various tissues. Therefore, by administering to a subject a nuclear protein or a fragment peptide thereof that has an activity of mobilizing mesenchymal stem cells to peripheral blood, the mesenchymal stem cells are recruited into the peripheral blood, and therapeutic effects for various diseases may be provided by the mesenchymal stem cells' anti-inflammatory action, immunomodulatory action, anti-fibrotic action, and tissue regeneration-promoting action (due to the differentiation and/or anti-inflammatory action of mesenchymal stem cells).

The present application provides compositions containing a nuclear protein or a fragment peptide thereof for use in mobilizing mesenchymal stem cells into peripheral blood.

The compositions for use in mobilizing mesenchymal stem cells into the peripheral blood of the present application can be used as a pharmaceutical composition or a reagent composition. In the present application, the term “pharmaceutical composition” is used interchangeably with “medicament”, “drug” or “pharmacological composition”, and the term “reagent composition” is used interchangeably with “reagent”.

The compositions for use in mobilizing mesenchymal stem cells into peripheral blood of the present application can be used for treating a disease or pathological condition in a subject, for example, by mobilizing mesenchymal stem cells into peripheral blood.

Mesenchymal stem cells mobilized into peripheral blood using the composition for mobilizing mesenchymal stem cells into the peripheral blood of the present application can also be collected from the body, concentrated, and then used for treatment of a disease or pathological condition in a subject. The present application also provides use of a nuclear protein or a fragment thereof in the manufacture of a medicament or a reagent for collecting mesenchymal stem cells from the body.

The compositions for use in mobilizing mesenchymal stem cells into the peripheral blood of the present application can also be used in, for example, basic research, clinical research and such. Basic research and clinical research include, but are not limited to, mesenchymal stem cell mobilization research in vitro and mesenchymal stem cell mobilization research in laboratory animals. The present application also provides use of nuclear proteins or fragment peptides thereof in the manufacture of pharmaceuticals or reagents for basic or clinical research.

The compositions for use in mobilizing mesenchymal stem cells into peripheral blood can comprise one or more nuclear proteins, one or more fragment peptides, or a combination thereof.

In the present application, “mesenchymal stem cells” are cells that are collected from bone marrow or other tissues (blood such as umbilical cord blood, and skin, fat, pulp, etc.), can be cultured and proliferated on culture dishes (plastic or glass) as adherent cells, and have the ability to differentiate into mesenchymal tissues such as bone, cartilage, fat, and muscle. In one embodiment, mesenchymal stem cells also have the ability to differentiate into epithelial tissues and nerve tissues. In one embodiment, mesenchymal stem cells are cells capable of forming colonies. In the present application, mesenchymal stem cells may exist as a heterogeneous cell population comprising not only stem cells in the narrow sense (cells having self-renewal ability and differentiation ability) but also progenitor cells. Under culture conditions, the mesenchymal stem cells may include stem cells in the narrow sense, or may even include differentiated cells in addition to stem cells in the narrow sense and progenitor cells. In one embodiment, the mesenchymal stem cells may be composed only of stem cells in the narrow sense.

In the present application, progenitor cells are defined as cells with a unidirectional ability to differentiate into cells of specific tissues other than the blood system, and include cells that have the ability to differentiate into mesenchymal tissues, epithelial tissues, nerve tissues, parenchymatous organs, vascular endothelium.

In the present application, the mesenchymal stem cells include, but are not limited to, bone marrow mesenchymal stem cells and bone marrow-derived mesenchymal stem cells. The “bone marrow mesenchymal stem cells” exist in the bone marrow, and may be harvested from bone marrow and cultured and proliferated as adherent cells on culture dish (made of plastic or glass); and they are cells characterized in having the ability to differentiate into mesenchymal tissues such as bone, cartilage, fat, muscle and such. In one embodiment, bone marrow mesenchymal stem cells also have the ability to differentiate into epithelial tissues and nerve tissues. In one embodiment, bone marrow mesenchymal stem cells are cells capable of forming colonies. In the present application, the term “bone marrow mesenchymal stem cell” is used interchangeably with “bone marrow mesenchymal stromal cell”, “bone marrow pluripotent stem cell” or “bone marrow pluripotent stromal cell”.

“Bone marrow-derived mesenchymal stem cells” refers to bone marrow mesenchymal stem cells that have been mobilized from bone marrow to the outside of the bone marrow, and are cells that can be collected by peripheral blood collection, and further from mesenchymal tissues such as fat, epithelial tissues such as skin, or nerve tissues such as the brain. In the present application, the term “bone marrow-derived mesenchymal stem cell” can be used interchangeably with “bone marrow-derived mesenchymal stromal cell”, “bone marrow-derived pluripotent stem cell” or “bone marrow-derived pluripotent stromal cell”.

In one embodiment, bone marrow mesenchymal stem cells and bone marrow-derived mesenchymal stem cells are also characterized in that, by being administered to an injured part of a living body directly after collection or after once attached to a culture dish, the cells are also capable of differentiating into, for example, epithelial tissues such as skin-constituting keratinocytes or tissues of the nerve system which constitutes the brain.

Bone marrow mesenchymal stem cells and bone marrow-derived mesenchymal stem cells preferably have the ability to differentiate into osteoblast cells (identifiable by calcium deposition observed when differentiation is induced), cartilage cells (identifiable by being Alcian blue staining-positive, safranin-O staining-positive, or such), and fat cells (identifiable by being Sudan III staining-positive or such), and also differentiate into, for example, mesenchymal cells such as fibroblasts, smooth muscle cells, skeletal muscle cells, stromal cells, and tendon cells, nerve cells, pigment cells, epidermal cells, hair follicle cells (expressing cytokeratin family, hair keratin family or such), epithelial cells (for example, epithelial keratinized cells and intestinal epithelial cells express cytokeratin family or such), endothelial cells, and further differentiate into cells of parenchymal organs such as liver, kidney and pancreas, but the differentiated cells are not limited to the above cells.

Human mesenchymal stem cell markers can be exemplified by some or all of PDGFRα positive, PDGFRβ positive, Lin negative, CD45 negative, CD44 positive, CD90 positive, CD29 positive, Flk-1 negative, CD105 positive, CD73 positive, CD90 positive, CD71 positive, Stro-1 positive, CD106 positive, CD166 positive, CD31 negative, CD271 positive, and CD11b negative, but are not limited thereto.

Murine mesenchymal stem cell markers can be exemplified by some or all of CD44 positive, PDGFRα positive, PDGFRβ positive, CD45 negative, Lin negative, Sca-1 positive, c-kit negative, CD90 positive, CD105 positive, CD29 positive, Flk-1 negative, CD271 positive, and CD11b negative, but are not limited thereto.

Rat mesenchymal stem cell markers can be exemplified by some or all of PDGFRα positive, CD44 positive, CD54 positive, CD73 positive, CD90 positive, CD105 positive, CD29 positive, CD271 positive, CD31 negative, and CD45 negative, but are not limited thereto.

In the present application, examples of mesenchymal stem cells include PDGFRα-positive mesenchymal stem cells, PDGFRα-positive bone marrow-derived mesenchymal stem cells, and PDGFRα-positive bone marrow-derived cells obtained as adherent cells by cell culture of a mononuclear cell fraction in blood obtained by bone marrow harvest (bone marrow cell collection) or peripheral blood collection, but they are not limited thereto. Examples of PDGFRα-positive mesenchymal stem cells include PDGFRα- and CD44-positive cells, PDGFRα- and CD90-positive cells, PDGFRα- and CD105-positive cells, PDGFRα- and CD29-positive cells, and such. In one embodiment, PDGFRα-positive mesenchymal stem cells may be CD44-negative cells.

The present application provides compositions for use in the treatment of a disease or a pathological condition in a subject by mobilizing mesenchymal stem cells into the peripheral blood, comprising a nuclear protein or a fragment peptide thereof.

The compositions for use in the treatment of a disease or a pathological condition in a subject by mobilizing mesenchymal stem cells into the peripheral blood in the present application can be used as pharmaceutical compositions.

The subject in the present application is not particularly limited, and examples thereof include mammals, birds, and fish. Mammals include human and non-human animals, for example, human, mouse, rat, monkey, pig, dog, rabbit, hamster, guinea pig, horse, sheep, and whale, but are not limited thereto. In the present application, the term “subject” is used interchangeably with “patient”, “individual”, or “animal”.

The composition for use in the treatment of a disease or pathological condition in a subject by mobilization of mesenchymal stem cells into the peripheral blood in the present application can comprise one or more nuclear proteins, one or more fragment peptides, or combinations thereof.

In the present application, the treatment of a disease or pathological condition is selected from, for example, inflammation-suppressing therapy, immunomodulatory therapy, tissue regeneration-inducing therapy, and tissue fibrosis-suppressing therapy, but is not limited thereto.

In the present application, the disease or pathological condition is selected from inflammatory diseases, autoimmune diseases, diseases accompanied by tissue damage, ischemia, or necrosis, and fibrotic diseases, but is not limited thereto.

In the present application, the inflammatory disease or autoimmune disease is, for example, selected from inflammatory bowel disease and psoriasis, but is not limited thereto. The fibrotic disease is selected from, for example, lung fibrosis, liver fibrosis, and liver cirrhosis, but is not limited thereto. The disease accompanied by tissue damage, ischemia, or necrosis includes, for example, inflammatory bowel disease, but is not limited thereto. The inflammatory bowel disease includes, but is not limited to, ulcerative colitis and Crohn's disease.

In the present application, the term “nuclear protein” refers to a protein that exerts a certain function in the nucleus and is a protein other than 1) to 6) below:
1) High mobility group box 1 (HMGB 1) protein;
2) High mobility group box 2 (HMGB2) protein;
3) High mobility group box 3 (HMGB3) protein;
4) S100 calcium-binding protein A8 (S100A8) protein;
5) S100 calcium-binding protein A9 (S100A9) protein; and
6) Interleukin-1 (IL-1) family cytokines. In one embodiment, the nuclear protein of the present application is a protein that has the activity of mobilizing mesenchymal stem cells into peripheral blood.

In the present application, the term “activity of mobilizing mesenchymal stem cells into peripheral blood” is used interchangeably with “activity to increase the abundance of mesenchymal stem cells in peripheral blood”.

The nuclear protein in the present application includes, but is not limited to, for example, a nuclear protein involved in transcriptional regulation. A “protein involved in transcriptional regulation” refers to, among nuclear proteins, a protein having a function of regulating any process in transcription and includes, for example, transcription factors and transcription cofactors, but is not limited thereto.

In the present application, the transcription factor is a protein that controls transcription by binding to DNA by itself or in the form of a complex with other proteins, and includes general transcription factors (proteins that constitute a transcription apparatus), transcriptional regulation factors, transcription elongation factors, factors that regulate transcription by involvement in the transcription termination process, and such.

In the present application, the transcription cofactor is a protein that regulates transcription via protein-protein interactions without binding directly to DNA, and includes but is not limited to co-activators and co-repressors, which regulate transcription by intervening between (binding to both) a transcriptional regulatory factor and a general transcription factor.

In the present application, the “fragment peptide of a nuclear protein” refers to a fragment peptide derived from the above-mentioned nuclear protein. In one embodiment, a fragment peptide of a nuclear protein is a fragment that has an activity of mobilizing mesenchymal stem cells into the peripheral blood.

Fragment peptides of the HMGB1 protein, fragment peptides of the HMGB2 protein, fragment peptides of the HMGB3 protein, fragment peptides of the S100A8 protein, fragment peptides of the S100A9 protein, and fragment peptides of the IL-1 family cytokines are excluded from the fragment peptides of nuclear proteins in the present application.

In the present application, the term “a fragment peptide of a nuclear protein” is used interchangeably with “a fragment peptide derived from a nuclear protein”, “a partial peptide derived from a nuclear protein”, “a fragment peptide consisting of a portion of a nuclear protein”, “a partial peptide consisting of a portion of a nuclear protein”, or “a partial peptide of a nuclear protein”.

The activity of a nuclear protein or a fragment peptide thereof in the present application to mobilize mesenchymal stem cells into the peripheral blood can be assessed by i) collecting peripheral blood from an individual administered with a nuclear protein or a fragment peptide thereof and an individual not administered with the nuclear protein or fragment peptide, seeding and culturing in a culture dish (several days to 10 days), and counting the number of colonies formed; and ii) confirming that the formed colonies have the ability to adhere to the solid phase and proliferate (self-renewal ability), and the ability to differentiate into osteoblasts, chondrocytes and adipocytes. In i) above, before seeding the collected peripheral blood on a culture dish, red blood cells may be removed from the peripheral blood in a desired manner.

The nuclear protein or fragment peptide thereof in the present application can be obtained as a recombinant by incorporating DNA encoding it into an appropriate expression system, or it can be artificially synthesized. Thus, nuclear proteins or fragment peptides thereof in this application include nuclear proteins and fragment peptides thereof prepared using cells, and artificially synthesized nuclear proteins and fragment peptides thereof (i.e., artificial (synthetic) nuclear proteins and fragment peptides thereof).

In order to obtain the nuclear protein or fragment peptide thereof in the present application by genetic engineering techniques, DNA encoding the peptide may be incorporated into an appropriate expression system and expressed.

Hosts applicable to the present application include, but are not limited to, prokaryotic cells and eukaryotic cells. In addition, hosts applicable to the present application also include bacteria (e.g., Escherichia coli), yeast, animal cells (e.g., mammalian cells such as HEK293 cells and CHO cells, insect cells such as silkworm cells), plant cells and such, but are not limited thereto.

As the host/vector system applicable to the present application, for example, the expression vector pGEX and Escherichia coli can be shown. pGEX can express a foreign gene as a fusion protein with glutathione S-transferase (GST) (Gene, 67: 31-40, 1988). As such, pGEX into which DNA encoding the nuclear protein or fragment peptide thereof of the present application has been incorporated is introduced into an E. coli strain such as BL21 by heat shock, and after culturing for an appropriate period of time, isopropylthio-β-D-galactoside (IPTG) is added to induce the expression of the GST fusion peptide. GST in the present application adsorbs to glutathione Sepharose 4B, and thus the expression product can be easily separated and purified by affinity chromatography.

In addition to this, the following can be applied as a host/vector system for obtaining a genetic recombinant of the nuclear protein or fragment peptide thereof of the present application. First, when a bacterium is used as a host, a vector for expressing a fusion protein using a tag or such is commercially available. In addition, the genetic recombinant of the present application also includes those in which a tag or a partial peptide thereof is added.

The tag added to the nuclear protein or fragment peptide thereof of the present application is not particularly limited as long as it does not affect the activity of the nuclear protein or fragment peptide thereof of the present application, and includes, for example, a histidine tag (for example, 6×His, 10×His), HA tag, FLAG tag, GST tag, T7-tag, HSV-tag, E-tag, lck tag, and B-tag.

Among yeasts, it is known that Pichia yeast is effective for the expression of proteins having sugar chains. In terms of the addition of sugar chains, the expression system that uses a Baculovirus vector, which uses an insect cell as a host, is also useful (Bio/Technology, 6:47-55, 1988). Furthermore, mammalian cells are used for transfection with a vector that uses the promoter of CMV, RSV, SV40, or such. These host/vector systems can be used as an expression system for the nuclear proteins or fragment peptides thereof of the present application. In addition, plasmid vectors, retrovirus vectors, lentivirus vectors, adenovirus vectors, adeno-associated virus vectors, Sendai virus vectors, Sendai virus envelope vectors, papilloma virus vectors, and such virus vectors may also be used to introduce the gene, without limitation thereto. The vector may also contain a promoter DNA sequence that effectively induces gene expression, factors that control gene expression, and molecules necessary to maintain the stability of the DNA.

The resulting nuclear proteins or fragment peptides thereof in the present application can be isolated from the host cell or outside of the cell (such as medium), and purified as a substantially pure homogeneous protein. For separation and purification of proteins, any separation and purification methods used in standard protein purification may be utilized, without limitation. For example, chromatography columns, filters, ultrafiltration, salting out, solvent precipitation, solvent extraction, distillation, immunoprecipitation, SDS-polyacrylamide gel electrophoresis, isoelectric focusing, dialysis, recrystallization, and such are appropriately selected and combined for protein separation and purification.

Chromatography includes, for example, affinity chromatography, ion exchange chromatography, hydrophobic chromatography, gel filtration, reverse phase chromatography, adsorption chromatography and such (Marshak et al, Strategies for Protein Purification and Characterization: A Laboratory Course Manual. Ed Daniel R. Cold Spring Harbor Laboratory Press, 1996). These chromatographies can be performed using liquid chromatography such as HPLC and FPLC.

Further, the nuclear protein or fragment peptide thereof in the present application is preferably a substantially purified peptide. Here, “substantially purified” means that the degree of purification of the nuclear protein or fragment peptide thereof of the present application (the ratio of the nuclear protein or fragment peptide thereof of the present application in the entire protein component) is 50% or more, 60% or more, 70% or more, 80% or more, 90% or more, 95% or more, 100% or nearly 100%. The nearly 100% upper limit depends on the purification techniques and analysis techniques of those skilled in the art and may be, for example, 99.999%, 99.99%, 99.9%, or 99%.

Further, the substantially purified nuclear protein or fragment peptide thereof includes those purified by whatever purification method as long as they have the above purity. Examples include, but are not limited to, nuclear proteins and fragment peptides thereof substantially purified by appropriately selecting or combining the above-mentioned chromatography columns, filters, ultrafiltration, salting out, solvent precipitation, solvent extraction, distillation, immunoprecipitation, SDS-polyacrylamide gel electrophoresis, isoelectric focusing, dialysis, recrystallization and such.

On the other hand, the nuclear proteins or fragment peptides thereof in the present application can also be artificially synthesized. In the peptide synthesis method of the present application, peptides can be chemically synthesized by methods such as a peptide liquid-phase synthesis method and a peptide solid-phase synthesis method. The peptide solid-phase synthesis method is one of the methods generally used when chemically synthesizing a peptide. Polystyrene polymer gel beads having a diameter of about 0.1 mm modified with amino groups on the surface are used as a solid phase, from which the amino acid chain is extended one by one via dehydration reaction. When the sequence of the target peptide is completed, it is excised from the solid phase surface to obtain the target substance. Solid phase synthesis enables synthesis of ribosome peptides, which are difficult to synthesize in bacteria, introduction of unnatural amino acids such as D-form and stable isotope (²H, ¹³C, ¹⁵N, etc.)-substituted amino acids, introduction of heavy atom-substituted amino acids (e.g., selenoamino acids such as selenomethionine), modification of peptide and protein main chains, and such. When synthesizing a long peptide chain of 70 to more than 100 amino acids in the solid phase method, it can be synthesized by ligating two peptide chains using the native chemical ligation method. The nuclear protein or fragment peptide thereof in the present application may be in the form of a pharmaceutically acceptable salt of the protein or peptide. Examples of pharmaceutically acceptable salts include, but are not limited to, hydrochlorides, acetates, and trifluoroacetates. The nuclear protein or fragment peptide thereof in the present application may be in the form of a solvate of the protein or peptide, or a solvate of a pharmaceutically acceptable salt of the protein or peptide. A solvate refers to a solute molecule to which an arbitrary number of solvent molecules are coordinated, and examples thereof include hydrates, but are not limited thereto.

The amino acid length of the nuclear protein or fragment peptide thereof in the present application includes, for example, 25 to 35 amino acids, 20 to 40 amino acids, 10 to 50 amino acids, 10 to 70 amino acids, and 10 to 100 amino acids, but is not limited thereto.

Examples of the nuclear protein or fragment peptide thereof in the present application include nuclear proteins selected from below or fragment peptides derived therefrom:
1. BTF3 protein (Basic transcription factor 3);
2. SUPT16H protein (Suppressor of Ty 16 Homolog; or Facilitates chromatin transcription complex subunit SPT16);
3. YBX1 protein (Y-Box binding protein 1; or Nuclease-sensitive element-binding protein 1);
4. NPM1 protein (Nucleophosmin 1);
5. PA2G4 protein (Proliferation-associated protein 2G4);
6. PFDN5 protein (Prefoldin subunit 5);
7. PSMC3 protein (Proteasome (Prosome, Macropain) 26S subunit, ATPase 3; or 26S proteasome regulatory subunit 6A);
8. HNRNPK protein (Heterogeneous nuclear ribonucleoprotein K); and
9. Nuclear protein functionally equivalent to a protein selected from 1 to 8.

In light of the examples of the fragment peptides described in the Examples of the present application, the nuclear protein selected from 1 to 8 above is considered to have an activity of mobilizing mesenchymal stem cells to peripheral blood. Therefore, “functionally equivalent” as described in 9 above means functionally equivalent in terms of the activity of mobilizing mesenchymal stem cells to peripheral blood. Therefore, the nuclear protein described in 9 above can be expressed as a nuclear protein having an activity equivalent to that of the protein selected from 1 to 8 (equivalent activity to mobilize mesenchymal stem cells to peripheral blood). The fragment peptide derived from the nuclear protein selected from 1 to 9 above is a fragment peptide having an activity of mobilizing mesenchymal stem cells to peripheral blood.

Since the nuclear protein selected from 1 to 9 above or a fragment peptide thereof has an activity of mobilizing mesenchymal stem cells to peripheral blood, it is considered to have the effect of mobilizing mesenchymal stem cells to peripheral blood, as well as therapeutic effects on inflammatory diseases, autoimmune diseases, diseases accompanied by tissue damage, ischemia, or necrosis, and fibrotic diseases.

In the present application, the nuclear protein or a fragment peptide thereof includes, for example, a nuclear protein selected from below or a fragment peptide derived therefrom:
(I) a nuclear protein comprising an amino acid sequence selected from SEQ ID NOs: 1 to 34;
(II) a nuclear protein consisting of an amino acid sequence selected from SEQ ID NOs: 1 to 34;
(III) a nuclear protein comprising an amino acid sequence resulting from substitution, deletion, insertion, or addition of one or more amino acids in an amino acid sequence selected from SEQ ID NOs: 1 to 34;
(IV) a nuclear protein consisting of an amino acid sequence resulting from substitution, deletion, insertion, or addition of one or more amino acids in an amino acid sequence selected from SEQ ID NOs: 1 to 34;
(V) a nuclear protein comprising an amino acid sequence having about 80% or more sequence identity with an amino acid sequence selected from SEQ ID NOs: 1 to 34;
(VI) a nuclear protein consisting of an amino acid sequence having about 80% or more sequence identity with an amino acid sequence selected from SEQ ID NOs: 1 to 34;
(VII) a nuclear protein encoded by a DNA consisting of a nucleotide sequence selected from SEQ ID NOs: 57 to 90; and
(VIII) a nuclear protein encoded by a DNA that hybridizes under stringent conditions with a DNA consisting of a nucleotide sequence selected from SEQ ID NOs: 57 to 90.

In the present application, the fragment peptide of a nuclear protein includes, for example, a fragment peptide selected from below:
(i) a nuclear protein fragment peptide consisting of a portion of an amino acid sequence selected from SEQ ID NOs: 1 to 34;
(ii) a nuclear protein fragment peptide comprising an amino acid sequence selected from SEQ ID NOs: 35-56;
(iii) a nuclear protein fragment peptide consisting of an amino acid sequence selected from SEQ ID NOs: 35 to 56;
(iv) a nuclear protein fragment peptide consisting of a portion of an amino acid sequence selected from SEQ ID NOs: 35 to 56;
(v) a nuclear protein fragment peptide comprising an amino acid sequence resulting from substitution, deletion, insertion, or addition of one or more amino acids in an amino acid sequence selected from SEQ ID NOs: 35 to 56;
(vi) a nuclear protein fragment peptide consisting of an amino acid sequence resulting from substitution, deletion, insertion, or addition of one or more amino acids in an amino acid sequence selected from SEQ ID NOs: 35 to 56;
(vii) a nuclear protein fragment peptide comprising an amino acid sequence having about 80% or more sequence identity with an amino acid sequence selected from SEQ ID NOs: 35 to 56;
(viii) a nuclear protein fragment peptide consisting of an amino acid sequence having about 80% or more sequence identity with an amino acid sequence selected from SEQ ID NOs: 35 to 56;
(ix) a nuclear protein fragment peptide encoded by a DNA consisting of a nucleotide sequence selected from SEQ ID NOs: 91 to 112; and
(x) a nuclear protein fragment peptide encoded by a DNA that hybridizes under stringent conditions with a DNA consisting of a nucleotide sequence selected from SEQ ID NOs: 91 to 112.

In the present application, “plurality” includes, for example, 1 to 10, 1 to 9, 1 to 8, 1 to 7, 1 to 6, 1 to 5, 1 to 4, 1 to 3, or 1 or 2.

In the present application, “about 80% or more” means, for example, about 85% or more, about 90% or more, about 91% or more, about 92% or more, about 93% or more, about 94% or more, about 95% or more, about 96% or more, about 97% or more, about 98% or more, or about 99% or more.

In the present application, the “stringent conditions” can be shown as conditions of, for example, hybridization at 6×SSC, 40% formamide, 25° C., and washing at 1×SSC, 55° C. Stringency is affected by conditions such as salt concentration, formamide concentration, or temperature, and those skilled in the art can set these conditions to obtain the required stringency.

When the hybridization is carried out under stringent conditions, a DNA having a high homology in terms of nucleotide sequence is selected, and the possibility is increased for the protein isolated as a result to comprise a protein functionally equivalent to (e.g., homologue) a protein consisting of an amino acid sequence selected from SEQ ID NOs: 1 to 34, or to comprise a fragment peptide functionally equivalent to a fragment peptide consisting of an amino acid sequence selected from SEQ ID NOs: 35 to 56. A nucleotide sequence having high homology can exhibit, for example, about 60% or more, about 70% or more, or about 80% or more identity.

Further, in the present application, the fragment peptide of a nuclear protein includes, for example, a fragment peptide selected from below:
(1) a fragment peptide consisting of a portion of an amino acid sequence selected from SEQ ID NOs: 1 to 2, and which is a fragment peptide comprising the amino acid sequence described in SEQ ID NO: 35;
(2) a fragment peptide consisting of a portion of the amino acid sequence described in SEQ ID NO: 3, and which is a fragment peptide comprising the amino acid sequence described in SEQ ID NO: 36;
(3) a fragment peptide consisting of a portion of the amino acid sequence described in SEQ ID NO: 4, and which is a fragment peptide comprising an amino acid sequence selected from SEQ ID NOs: 37 to 39;
(4) a fragment peptide consisting of a portion of an amino acid sequence selected from SEQ ID NOs: 5 to 7, and which is a fragment peptide comprising an amino acid sequence selected from SEQ ID NOs: 40 to 41;
(5) a fragment peptide consisting of a portion of the amino acid sequence described in SEQ ID NO: 8, and which is a fragment peptide comprising the amino acid sequence described in SEQ ID NO: 42;
(6) a fragment peptide consisting of a portion of the amino acid sequence described in SEQ ID NO: 9, and which is a fragment peptide comprising the amino acid sequence described in SEQ ID NO: 43;
(7) a fragment peptide consisting of a portion of the amino acid sequence described in SEQ ID NO: 10, and which is a fragment peptide comprising the amino acid sequence described in SEQ ID NO: 44;
(8) a fragment peptide consisting of a portion of an amino acid sequence selected from SEQ ID NOs: 11 to 16, and which is a fragment peptide comprising the amino acid sequence described in SEQ ID NO: 45;
(9) a fragment peptide consisting of a portion of an amino acid sequence selected from SEQ ID NOs: 17 to 18, and which is a fragment peptide comprising the amino acid sequence described in SEQ ID NO: 46;
(10) a fragment peptide consisting of a portion of the amino acid sequence described in SEQ ID NO: 19, and which is a fragment peptide comprising the amino acid sequence described in SEQ ID NO: 47;
(11) a fragment peptide consisting of a portion of the amino acid sequence described in SEQ ID NO: 20, and which is a fragment peptide comprising an amino acid sequence selected from SEQ ID NOs: 48 to 50;
(12) a fragment peptide consisting of a portion of an amino acid sequence selected from SEQ ID NOs: 21, 23, and 24, and which is a fragment peptide comprising the amino acid sequence described in SEQ ID NO: 51;
(13) a fragment peptide consisting of a portion of an amino acid sequence selected from SEQ ID NOs: 21 to 26, and which is a fragment peptide comprising the amino acid sequence described in SEQ ID NO: 52;
(14) a fragment peptide consisting of a portion of the amino acid sequence described in SEQ ID NO: 27, and which is a fragment peptide comprising the amino acid sequence described in SEQ ID NO: 53;
(15) a fragment peptide consisting of a portion of an amino acid sequence selected from SEQ ID NOs: 28 to 29, and which is a fragment peptide comprising the amino acid sequence described in SEQ ID NO: 54;
(16) a fragment peptide consisting of a portion of the amino acid sequence described in SEQ ID NO: 30, and which is a fragment peptide comprising the amino acid sequence described in SEQ ID NO: 55; and
(17) a fragment peptide consisting of a portion of an amino acid sequence selected from SEQ ID NOs: 31 to 34, and which is a fragment peptide comprising the amino acid sequence described in SEQ ID NO: 56.

The nuclear protein selected from (I) to (VIII) above is a nuclear protein having an activity of mobilizing mesenchymal stem cells into peripheral blood. Further, the fragment peptides derived from a nuclear protein selected from (I) to (VIII) above, nuclear protein fragment peptides selected from (i) to (x) above, and nuclear protein fragment peptides selected from (1) to (17) above are fragment peptides having an activity of mobilizing mesenchymal stem cells into peripheral blood. Therefore, these nuclear proteins and fragment peptides are considered to have the effect of mobilizing mesenchymal stem cells into peripheral blood, as well as therapeutic effects on inflammatory diseases, autoimmune diseases, diseases accompanied by tissue damage, ischemia, or necrosis, and fibrotic diseases.

The present application also provides nuclear proteins selected from 1 to 9 above or fragment peptides derived therefrom, nuclear proteins selected from (I) to (VIII) above or fragment peptides derived therefrom, nuclear protein fragment peptides selected from (i) to (x) above, and nuclear protein fragment peptides selected from (1) from (17) above.

The amino acid sequences described in SEQ ID NOs: 1 to 56 are amino acid sequences of the proteins or peptides shown in Tables 1-1 and 1-2 below.

TABLE 1-1

Position

| | | in the |
|---|---|---|
| SEQ | | full-length |
| ID NO: | Name | protein |
| 1 | Mouse BTF3 protein isoform 1 | |
| 2 | Mouse BTF3 protein isoform 2 | |
| 3 | Mouse SUPT16H protein | |
| 4 | Mouse YBX1 protein | |
| 5 | Mouse NPM1 protein isoform 1 | |
| 6 | Mouse NPM1 protein isoform 2 | |
| 7 | Mouse NPM1 protein isoform 3 | |
| 8 | Mouse PA2G4 protein | |
| 9 | Mouse PFDN5 protein | |
| 10 | Mouse PSMC3 protein | |
| 11 | Mouse HNRNPK protein isoform 1 | |
| 12 | Mouse HNRNPK protein isoform 2 | |
| 13 | Mouse HNRNPK protein isoform 3 | |
| 14 | Mouse HNRNPK protein isoform 4 | |
| 15 | Mouse HNRNPK protein isoform 5 | |
| 16 | Mouse HNRNPK protein isoform 6 | |
| 17 | Human BTF3 protein isoform A | |
| 18 | Human BTF3 protein isoform B | |
| 19 | Human SUPT16H protein | |
| 20 | Human YBX1 protein | |
| 21 | Human NPM1 protein isoform 1 | |
| 22 | Human NPM1 protein isoform 2 | |
| 23 | Human NPM1 protein isoform 3 | |
| 24 | Human NPM1 protein isoform 4 | |
| 25 | Human NPM1 protein isoform 5 | |
| 26 | Human NPM1 protein isoform 6 | |
| 27 | Human PA2G4 protein | |

TABLE 1-2

| | | |
|---|---|---|
| 28 | Human PFDN5 protein isoform α | |
| 29 | Human PFDN5 protein isoform γ | |
| 30 | Human PSMC3 protein | |
| 31 | Human HNRNPK protein isoform a | |
| 32 | Human HNRNPK protein isoform b | |
| 33 | Human HNRNPK protein isoform c | |
| 34 | Human HNRNPK protein isoform d | |
| 35 | Mouse BTF3 peptide-1 | 63-92 |
| 36 | Mouse SUPT16H peptide-1 | 108-137 |
| 37 | Mouse YBX1 peptide-1 | 174-203 |
| 38 | Mouse YBX1 peptide-2 | 225-254 |
| 39 | Mouse YBX1 peptide-3 | 273-302 |
| 40 | Mouse NPM1 peptide-1 | 209-238 |
| 41 | Mouse NPM1 peptide-2 | 221-250 |
| 42 | Mouse PA2G4 peptide-1 | 43-72 |
| 43 | Mouse PFDN5 peptide-1 | 99-128 |
| 44 | Mouse PSMC3 peptide-1 | 55-84 |
| 45 | Mouse HNRNPK peptide-1 | 242-271 |
| 46 | Human BTF3 peptide-1 | 65-94 |
| 47 | Human SUPT16H peptide-1 | 108-137 |
| 48 | Human YBX1 peptide-1 | 176-205 |
| 49 | Human YBX1 peptide-2 | 227-256 |
| 50 | Human YBX1 peptide-3 | 275-304 |
| 51 | Human NPM1 peptide-1 | 210-240 |
| 52 | Human NPM1 peptide-2 | 223-252 |
| 53 | Human PA2G4 peptide-1 | 43-72 |
| 54 | Human PFDN5 peptide-1 | 99-128 |
| 55 | Human PSMC3 peptide-1 | 52-81 |
| 56 | Human HNRNPK peptide-1 | 242-271 |

The nucleotide sequences described in SEQ TD NOs: 57 to 112 are examples of the nucleotide sequences of DNAs encoding the proteins or peptides shown in Tables 2-1 and 2-2 below. Other DNA sequences encoding the proteins or peptides shown in Tables 2-1 and 2-2 below can be produced by a method of converting the amino acid residues of the proteins or peptides to corresponding codons using codon tables known to those skilled in the art (reverse translation). Reverse translation can be performed by using a variety of software (including programs, algorithms, etc.) developed for the analysis of amino acid and nucleic acid sequences as desired.

TABLE 2-1

| | |
|---|---|
| SEQ | Position |

| ID NO: | Name | in full |
|---|---|---|
| | | length DNA |
| 57 | Mouse BTF3 protein isoform 1 | |
| 58 | Mouse BTF3 protein isoform 2 | |
| 59 | Mouse SUPT16H protein | |
| 60 | Mouse YBX1 protein | |
| 61 | Mouse NPM1 protein isoform 1 | |
| 62 | Mouse NPM1 protein isoform 2 | |
| 63 | Mouse NPM1 protein isoform 3 | |
| 64 | Mouse PA2G4 protein | |
| 65 | Mouse PFDN5 protein | |
| 66 | Mouse PSMC3 protein | |
| 67 | Mouse HNRNPK protein isoform 1 | |
| 68 | Mouse HNRNPK protein isoform 2 | |
| 69 | Mouse HNRNPK protein isoform 3 | |
| 70 | Mouse HNRNPK protein isoform 4 | |
| 71 | Mouse HNRNPK protein isoform 5 | |
| 72 | Mouse HNRNPK protein isoform 6 | |
| 73 | Human BTF3 protein isoform A | |
| 74 | Human BTF3 protein isoform B | |
| 75 | Human SUPT16H protein | |
| 76 | Human YBX1 protein | |
| 77 | Human NPM1 protein isoform 1 | |
| 78 | Human NPM1 protein isoform 2 | |
| 79 | Human NPM1 protein isoform 3 | |
| 80 | Human NPM1 protein isoform 4 | |
| 81 | Human NPM1 protein isoform 5 | |
| 82 | Human NPM1 protein isoform 6 | |
| 83 | Human PA2G4 protein | |
| 84 | Human PFDN5 protein isoform α | |

TABLE 2-2

| | | |
|---|---|---|
| 85 | Human PFDN5 protein isoform γ | |
| 86 | Human PSMC3 protein | |
| 87 | Human HNRNPK protein isoform a | |
| 88 | Human HNRNPK protein isoform b | |
| 89 | Human HNRNPK protein isoform c | |
| 90 | Human HNRNPK protein isoform d | |
| 91 | Mouse BTF3 peptide-1 | 187-276 |
| 92 | Mouse SUPT16H peptide-1 | 322-411 |
| 93 | Mouse YBX1 peptide-1 | 520-609 |
| 94 | Mouse YBX1 peptide-2 | 673-762 |
| 95 | Mouse YBX1 peptide-3 | 817-906 |
| 96 | Mouse NPM1 peptide-1 | 625-714 |
| 97 | Mouse NPM1 peptide-2 | 661-750 |
| 98 | Mouse PA2G4 peptide-1 | 127-216 |
| 99 | Mouse PFDN5 peptide-1 | 295-384 |
| 100 | Mouse PSMC3 peptide-1 | 163-252 |
| 101 | Mouse HNRNPK peptide-1 | 724-813 |
| 102 | Human BTF3 peptide-1 | 193-282 |
| 103 | Human SUPT16H peptide-1 | 322-411 |
| 104 | Human YBX1 peptide-1 | 526-615 |
| 105 | Human YBX1 peptide-2 | 679-768 |
| 106 | Human YBX1 peptide-3 | 823-912 |
| 107 | Human NPM1 peptide-1 | 628-720 |
| 108 | Human NPM1 peptide-2 | 667-756 |
| 109 | Human PA2G4 peptide-1 | 127-216 |
| 110 | Human PFDN5 peptide-1 | 295-384 |
| 111 | Human PSMC3 peptide-1 | 154-243 |
| 112 | Human HNRNPK peptide-1 | 724-813 |

In the present application, the nuclear protein or a fragment peptide thereof is a nuclear protein or a fragment peptide thereof that comprises, for example, a nuclear localization signal (NLS). A nuclear localization signal (NLS) is an amino acid sequence having a certain pattern and has a function of transferring a protein/peptide having the amino acid sequence into the nucleus.

For example, many nuclear proteins are known to carry NLS in their amino acid sequences, and move into the nucleus by binding to the nuclear transport receptor (also referred to as nuclear transport proteins and nuclear transport factors) that recognizes the NLS.

Examples of currently known NLS (hereinbelow referred to as a known NLS) include those listed in Tables 3-1 and 3-2 below.

TABLE 3-1

| Type | | Sequence pattern | Specific example (source) |
|---|---|---|---|
| cNLS | monopartite | K-K/R-X-K/R (SEQ ID | PKKKRRV |
| (classical | | NO: 113) | (Lange et al., (2007) J. Biol. |
| NLS) | | | Chem., 282, 5101-5105; |
| | | | Kosugi et al., (2009) J. Biol. |
| | | | Chem., 284, 478-485., etc.) |
| | | | (SEQ ID NO: 114) |
| | bipartite | (K/R)(K/R)X₁₀₋₁₂(K/R)_{3/5} | KRPAATKKAGQAKKKK |
| | | ((K/R)_{3/5} = at least 3 amino | (Kosugi et at., (2009) J. Biol. |
| | | acids among 5 consecutive | Chem., 284, 478-485., |
| | | amino acids are K or R) (SEQ | etc.) (SEQ ID NO: 118) |
| | | ID NO: 115) | |
| | | KRX₁₀₋₁₂K(K/R)(K/R) (SEQ | |
| | | ID NO: 116) | |
| | | KRX₁₀₋₁₂K(K/R)X(K/R) (SEQ | |
| | | ID NO: 117) | |

| | | |
|---|---|---|
| PY-NLS | Those having the following 2 | FGNYNNQSSNFGPMKGGNF |
| | motifs: | GGRSSGPY |
| | 1) R/K/H-X₍₂₋₅₎-P-Y (SEQ ID | (Lee et al., (2006) Cell, 126, |
| | NO: 119) | 543-558; Süel et al., (2008) |
| | 2) φG/A/Sφφ X₍₁₁₋₁₃₎PY | PLoS Biol., 6, e137, etc.) |
| | (φ is an amino acid having | (SEQ ID NO: 121) |
| | hydrophobic | |
| | side-chain) (SEQ ID NO: 120) | |
| | Those having the following 2 | GEGERPAQNEKRKEKNIKRG |
| | motifs: | GNRFEPY |
| | 1) R/K/H-X₍₂₋₅₎-P-Y (SEQ ID | (Lee et al., (2006) Cell, 126, |
| | NO: 119) | 543-558; Süel et al., (2008) |
| | 2) basic-enriched₍₅₋₈₎X₍₈₋₁₀₎PY | PLoS Biol., 6, e137, etc.) |
| | (“basic-enriched” is a region | (SEQ ID NO: 123) |
| | rich in basic amino | |
| | acids) (SEQ ID NO: 122) | |

TABLE 3-2

| | | |
|---|---|---|
| BIB domain | Amino acid residues 32-74 | VHSHKKKKIRTSPTFRRPKTL |
| (β-like importin binding | of rpL23a protein | RLRRQPKYPRKSAPRRNKLD |
| domain) | | HY |
| | | (Jäkel, S. & Görlich, D. (1998) |
| | | EMBO J., 17, 4491-4502) (SEQ |
| | | ID NO: 124) |
| BIB domain like sequence | (1) MSHRKFSAPRHGSLG | Kimura et al., Mol. Cell. |
| | FLPRKRSSRHRGKVKS | Proteomics (2013), 12, |
| | FPKDDP (SEQ ID NO: | 145-157, FIG. 3C 2)-3) |
| | 125) | |
| | (2) EVTNDFVMLKGCVVG | |
| | TKKRVLTLRKSLLVQTK | |
| | RRALEKIDLKFIDTTSK | |
| | F (SEQ ID NO: 126) | |
| | (3) SLGQSASETEEDTVSV | |
| | SKKEKNRKRRNRKKK | |
| | KKPQRVRGVSSESSG | |
| | DREK (SEQ ID NO: 127) | |
| | (4) PTRYSVDIPLDKTVVN | |
| | KDVFRDPALKRKARRE | |
| | AKVKFEERYKTGKNK | |
| | WFF (SEQ ID NO: 128) | |
| | (5) KMFKGKRGAQLAKDIA | |
| | RRSKTFNPGAGLPTDK | |
| | KKGGPSPGDVEAIKNA | |
| | IA (SEQ ID NO: 129) | |

In addition, known NLS include sequences registered on the NLSdb database (<https://rostlab.org/services/nlsdb/>). The sequences registered on NLSdb can be viewed and downloaded on the above website. Among the NLS sequences registered on NLSdb, those whose annotation type is “Experimental” or “By Expert” can be estimated as having a function of translocating a protein/peptide into the nucleus, and are therefore treated as known NLS in the present application.

The NLS in the present application may be an NLS predicted by using a specific program (hereinbelow referred to as a predicted NLS). Whether a predicted NLS is contained in the desired amino acid sequence can be determined using the following program: SeqNLS (Lin et al., PLoS One. 2013 Oct. 29; 8 (10):e76864) or NLStradamus (Nguyen et al., BMC Bioinformatics. 2009 Jun. 29; 10:202).

In one embodiment, the NLS is a known NLS. In one embodiment, the NLS is a known NLS selected from the group consisting of cNLS, PY-NLS, BIB domain, and BIB domain-like sequences. In one embodiment, the cNLS is a monopartite cNLS. In one embodiment, the monopartite cNLS is KKEK (SEQ ID NO: 130).

An effective amount of a nuclear protein of the present application or a fragment peptide thereof, or a pharmaceutical composition comprising the same (hereinafter referred to as a pharmaceutical composition and such) is administered to a subject for the treatment of diseases and conditions described herein.

The effective amount in the present application means an amount sufficient for the treatment of the diseases or pathological conditions described herein. The treatment in the present application includes alleviation, delay, inhibition, amelioration, remission, cure, and full recovery, but are not limited thereto.

There is no limitation on the site of administration of the pharmaceutical composition and such of the present application, and the pharmaceutical composition and such of the present application can exert its effect when administered to any site, such as a site where the symptoms of the disease or pathological condition appear or a site nearby, a site different from these sites (sites other than these sites), a site separated from a site where the symptoms of the disease or pathological condition appear, a site distal to a site where the symptoms of the disease or pathological condition appear, or a site distal and ectopic to a site where the symptoms of the disease or pathological condition appear.

Further, the pharmaceutical composition and such of the present application can exert its effect when administered to any tissue, such as a tissue different from a tissue in which the symptoms of the disease or the pathological condition appear, a tissue separated from a tissue in which the symptoms of the disease or the pathological condition appear, a tissue distal to a tissue in which the symptoms of the disease or the pathological condition appear, or a tissue distal and ectopic to a tissue in which the symptoms of the disease or pathological condition appear.

Methods of administering the pharmaceutical composition and such of the present application include, but are not limited to, oral administration and parenteral administration, and methods of parenteral administration include intravascular administration (intra-arterial administration, intravenous administration, etc.), intramuscular administration, subcutaneous administration, intradermal administration, intraperitoneal administration, nasal administration, pulmonary administration, transdermal administration, and such. In addition, the pharmaceutical composition and such of the present application can be administered systemically or locally (for example, subcutaneously, intradermally, or to the skin surface, eyeball, or palpebral conjunctiva, nasal mucosa, oral and gastrointestinal mucosa, vaginal and endometrial mucosa, or injured site) by injection administration, for example, intravenous injection, intramuscular injection, intraperitoneal injection, and subcutaneous injection.

In place of the nuclear protein of the present application or a fragment peptide thereof, a cell secreting the nuclear protein or a fragment peptide thereof, a gene therapy vector into which a DNA encoding the nuclear protein or fragment peptide thereof is inserted, and a pharmaceutical composition comprising them can be used.

In addition, the administration method can be appropriately selected depending on the age and symptoms of a patient. When the pharmaceutical composition and such of the present application is administered, for example, the dose can be selected from the range of 0.0000001 mg to 1000 mg per kilogram of body weight per administration. Alternatively, for example, the dose can be selected from the range of 0.00001 to 100000 mg/body per patient. When administering cells secreting the nuclear protein of the present application or a fragment peptide thereof or gene therapy vectors into which DNA encoding the nuclear protein or a fragment peptide thereof is inserted, they can be administered so that the amount of the nuclear protein or fragment peptide thereof is within the above range. However, the pharmaceutical compositions in the present application are not limited to these dosages.

The pharmaceutical compositions of the present application can be formulated according to conventional methods (e.g., Remington's Pharmaceutical Science, latest edition, Mark Publishing Company, Easton, U.S.A.), and may contain pharmaceutically acceptable carriers or additives together. Examples include, but are not limited to, surfactants, excipients, coloring agents, perfumes, preservatives, stabilizers, buffers, suspending agents, isotonizing agents, binding agents, disintegrants, lubricants, fluidity-promoting agents, and flavoring agents. Other commonly used carriers can also be used as appropriate. Specific examples include light anhydrous silicic acid, lactose, crystalline cellulose, mannitol, starch, carmellose calcium, carmellose sodium, hydroxypropyl cellulose, hydroxypropyl methyl cellulose, polyvinylacetal diethylaminoacetate, polyvinylpyrrolidone, gelatin, medium-chain fatty acid triglyceride, polyoxyethylene hydrogenated castor oil 60, white sugar, carboxymethyl cellulose, cornstarch, and inorganic salts.

All prior art documents cited herein are incorporated herein as references.

Herein below, the present invention will be further illustrated with reference to Examples, but it is not to be construed as being limited thereto.

### EXAMPLES

### Example 1

### Mobilization of Mesenchymal Stem Cells by Fragment Peptides of Nuclear Proteins

### (1) Materials and Methods

### i) Peptide Production

Fragment peptides of the nuclear proteins shown in the table below were chemically synthesized by the solid phase method (all of the obtained peptides are in the form of trifluoroacetic acid (TFA) salts).

TABLE 4

| | Another name considering the | SEQ |
|---|---|---|
| Name | name of the protein origin | ID NO |
| NP-1 | Mouse BTF3 peptide-1 | 35 |
| NP-2 | Mouse SUPT16H peptide-1 | 36 |
| NP-3 | Mouse YBX1 peptide-1 | 37 |
| NP-4 | Mouse YBX1 peptide-2 | 38 |
| NP-5 | Mouse YBX1 peptide-3 | 39 |
| NP-6 | Mouse NPM1 peptide-1 | 40 |
| NP-7 | Mouse NPM1 peptide-2 | 41 |
| NP-8 | Mouse PA2G4 peptide-1 | 42 |
| NP-9 | Mouse PFDN5 peptide-1 | 43 |
| NP-10 | Mouse PSMC3 peptide-1 | 44 |
| NP-11 | Mouse HNRNPK peptide-1 | 45 |

### ii) Peptide Administration

C57BL/6J mice (8 weeks old, male, body weight 25 g) were prepared and divided into groups to which any of peptides NP-1 to NP-11 described in the above table were administered and a control group. The peptide was administered by injecting into the tail vein, a solution of each peptide adjusted to a concentration of 1 μg/μL using physiological saline as a solvent in an amount of 100 μL/animal (4 mg/kg dose of peptide). For the control group, physiological saline was injected into the tail vein in an amount of 100 μL/animal.

iii) Cell Collection from Peripheral Blood

Predetermined time after the administration of physiological saline or peptides NP-1 to NP-11 (NP-1 to NP-6 and NP-8: after 14 hours; NP-7, NP-10 and NP-11: after 16 hours; NP-9: after 24 hours), about 800 μL to 1000 μL of peripheral blood was collected from the hearts under general anesthesia (1 mL syringe containing heparin was used). To remove red blood cells, an equal volume of Hetasep (STEMCELL Technologies Inc., Cat No. ST-07906) as the collected blood was added and centrifuged for 2 min at 100G, incubated for 15 min at room temperature, and then the supernatant was collected. The supernatant was used in the next experiment as a sample containing nucleated cells in peripheral blood.

### iv) Colony Assay

The supernatant (sample containing peripheral blood-derived cells) obtained by the above procedure was seeded on collagen I-coated 6-well plates (Corning, Cat No. 356400), and cultured for 10 days under the conditions of 37° C., 5% CO₂ and 5% O₂, using a medium containing Expansion Medium prepared using the MesenCult Expansion Kit (STEMCELL Technologies, Cat No. ST-05513) according to the manual of the kit, 1% L-glutamine (Nacalai Tesque Inc.), 10 μM ROCK inhibitor (Y27632, Tocris Bioscience) and 1% penicillin/streptomycin (Nacalai Tesque Inc.) (all numerical values are final concentrations). The medium was replaced with fresh medium twice a week during the culture period. On the 10^{th} day of culture, cells on the plates were stained with a Differential Quik Stain Kit (Sysmex Corporation, Cat No. 16920), and the number of colonies containing 50 or more cells was counted.

In the experiments conducted by the present inventors so far, all the colonies obtained as a result of culturing peripheral blood on a solid phase such as a dish or a plate have adherability to the solid phase and have self-renewal ability. In addition, they have been confirmed to be PDGFRα positive, and have the ability to differentiate into bone, cartilage, fat, epithelium, and such.

In addition, the colonies obtained as a result of culturing, on a solid phase, peripheral blood after administration of a peptide consisting of amino acid residues 1-44 of the human HMGB1 protein (hereinafter, HMGB1 peptide 1-44), which has an activity of mobilizing mesenchymal stem cells into peripheral blood, have been confirmed to have adherability to solid phase and self-renewal ability, and to be PDGFRα positive. They have also been confirmed to have a gene expression profile characteristic to mesenchymal stem cells, based on the results of clustering the transcriptome analysis data and performing gene ontology analysis.

Furthermore, it has been confirmed that the number of colonies obtained by solid-phase culture is larger in the peripheral blood after administration of HMGB1 peptide 1-44 than in the peripheral blood after administration of physiological saline.

Therefore, the colonies obtained as a result of culturing peripheral blood on a solid phase are mesenchymal stem cells, and it is considered that an increase in the number of colonies detected in a solid phase culture of peripheral blood indicates an increase in the number of mesenchymal stem cells in the peripheral blood.

In addition, since usually mesenchymal stem cells are rarely present in peripheral blood, it is thought that the increased amount of mesenchymal stem cells was mobilized into peripheral blood from tissues other than peripheral blood (for example, bone marrow).

From the above, the number of colonies detected in the solid-phase culture of peripheral blood after administration of a test substance can be used as an indicator of the activity of the test substance to mobilize mesenchymal stem cells into peripheral blood.

### (2) Result

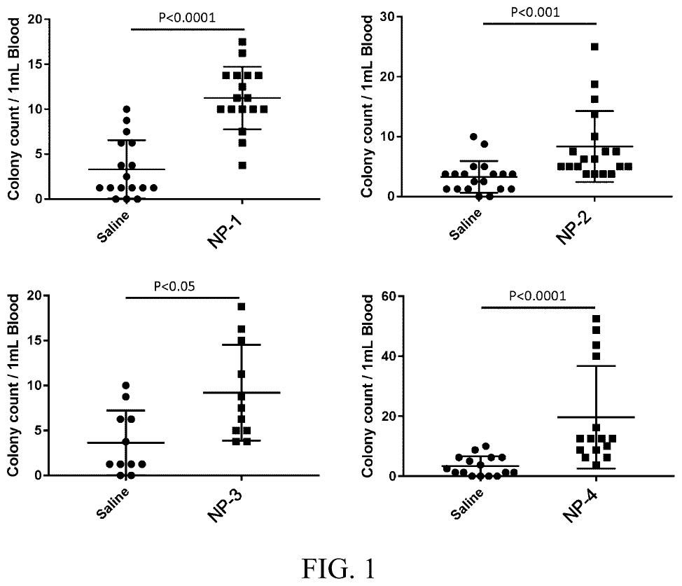

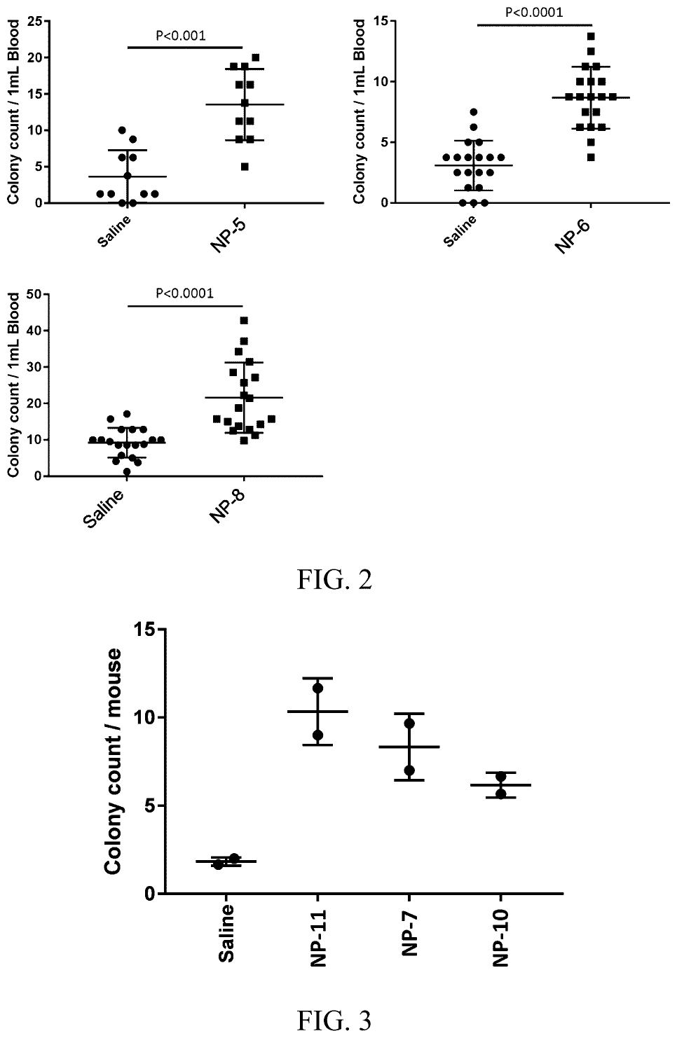

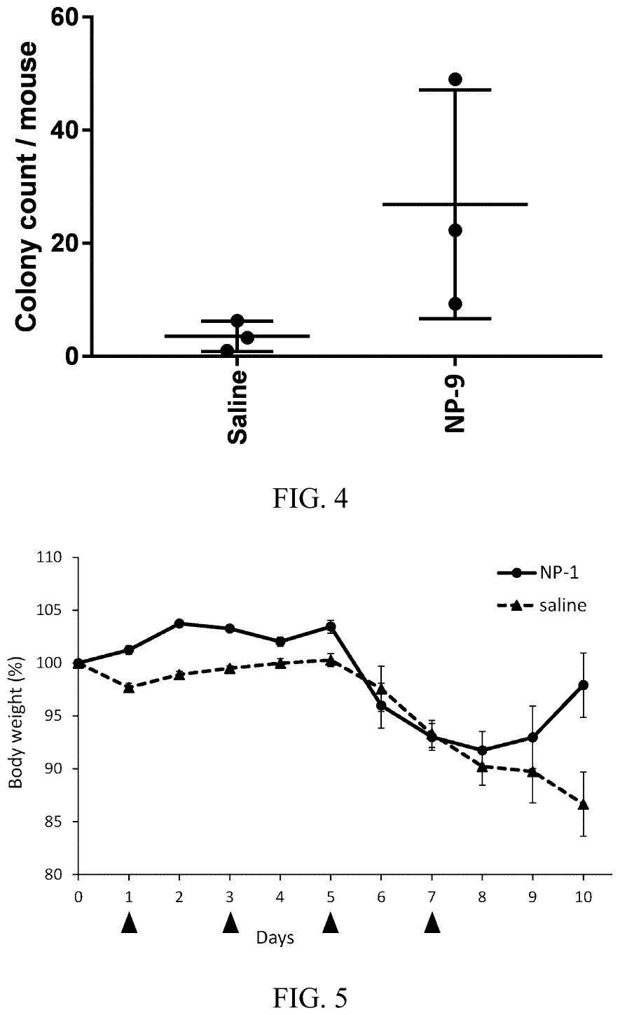

In the mice administered with any of the peptides NP-1 to NP-11, the number of colonies obtained on the plate by culturing the peripheral blood-derived cells was larger than that in the mice administered with physiological saline (FIGS. 1 to 4).

As described above, an increase in the number of colonies detected by the colony assay described herein indicates an increase in the number of mesenchymal stem cells in peripheral blood, and thus these results demonstrate that the fragment peptides of the nuclear proteins have the activity of mobilizing mesenchymal stem cells into peripheral blood.

### Example 2

### Efficacy of Nuclear Protein-Derived Peptides for Inflammatory Bowel Disease

### (1) Materials and Methods

### i) Drugs

Dextran sulfate sodium (DSS) (molecular weight 5,000-6,000, manufactured by Nacalai Tesque Inc., Catalog No. 10930-94) was dissolved in water to prepare a 2.5% (w/v) DSS aqueous solution. In addition, the peptides NP-1 to NP-4 (all TFA salts) described in Example 1 were used as test substances.

### ii) Generation of the Inflammatory Bowel Disease (IBD) Model Mice

C57BL/6J mice (8 weeks old, male, body weight about 20 g) were allowed free drinking of 2.5% DSS aqueous solution in place of purified water (RO water) to induce colitis (drinking of the DSS aqueous solution was continued for 10 days).

iii) Peptide Administration

The IBD model mice prepared as described above were divided into peptide administration groups (NP-1 to NP-4, each n=3) and a control group (n=3). Administration of each peptide was performed on Days 1, 3, 5 and 7 after the start of drinking the DSS aqueous solution, by adjusting the peptide solution to a concentration of 0.5 mg/mL using physiological saline as the solvent, and injecting the amount of 200 μL/animal (5 mg/kg dose of peptide) into the tail vein. In the control group, 200 μL/animal of physiological saline was injected into the tail vein on Days 1, 3, 5 and 7 after the start of drinking the DSS aqueous solution.

### iv) Evaluation of the Effect of Peptide Administration

Mice were weighed daily for 10 days from the start of drinking the DSS aqueous solution.

### (2) Result

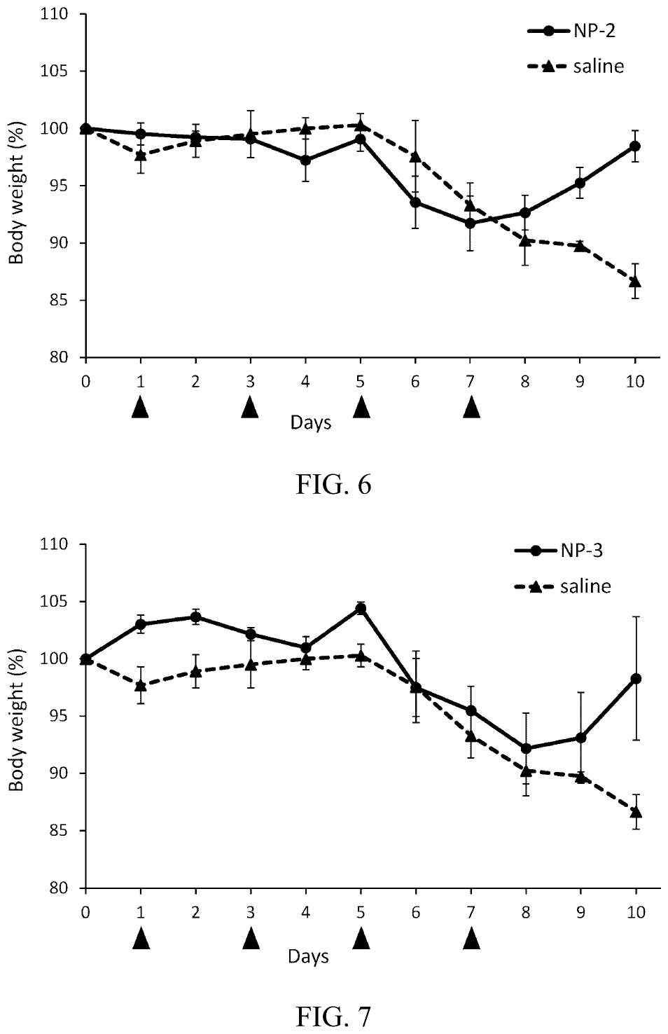

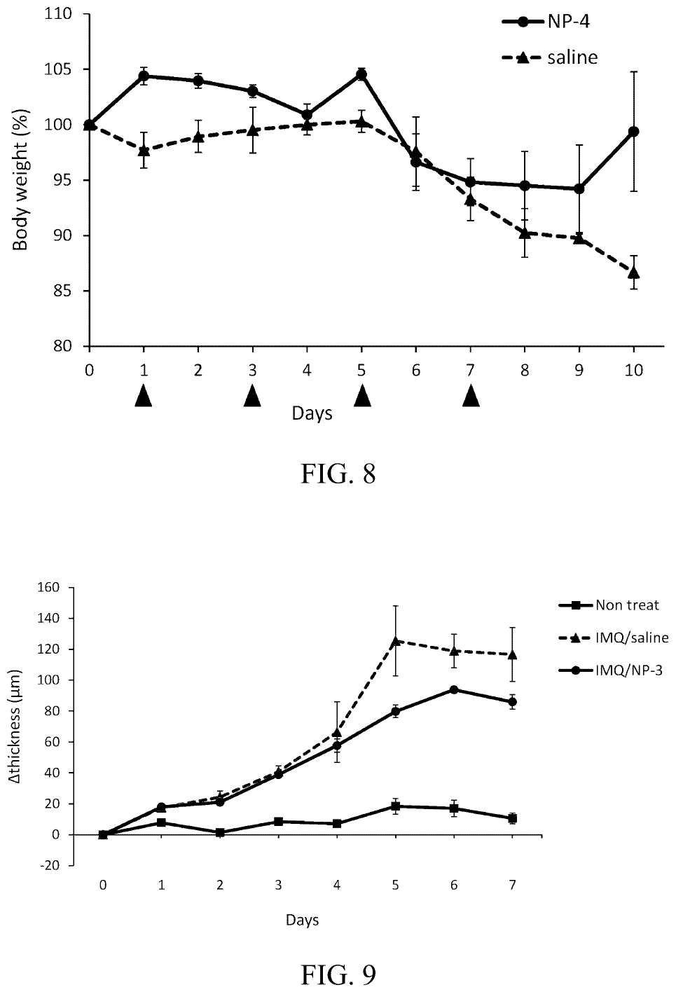

Changes in the body weight of mice during the test period are shown in FIGS. 5 to 8 (see “Saline” for the control group and “NP-1”, “NP-2”, “NP-3”, “NP-4” for the peptide NP-1 to NP-4 administration groups.). The body weight of the control group decreased with the passage of days, and was about 87% on the 10^{th} day after the start of drinking the DSS aqueous solution as compared with that before the start of the DSS drinking. On the other hand, in all of the peptide NP-1 to NP-4 administration groups, the body weight loss was suppressed as compared with the control group, and the body weight on the 10^{th} day after the start of drinking the DSS aqueous solution was significantly larger than that in the control group.

One of the symptoms of IBD is known to be occurrence of body weight loss, and the reason is thought to be malnutrition due to inflammation and tissue damage (such as ulcer) that occur in the intestinal mucosa. Furthermore, intravenous injection of mesenchymal stem cells in an animal IBD model is known to improve various symptoms including body weight loss, epithelial damage in the intestinal tract, infiltration of inflammatory cells, and such. Such improvement of symptoms is due to suppression of inflammation of the intestinal mucosa by the anti-inflammatory effect of mesenchymal stem cells, and resulting facilitation of mucosal tissue regeneration, and such.

This time, by administering fragment peptides of nuclear proteins of the present application to the IBD model mice, the body weight loss was suppressed. This is thought to be the result of mobilization of mesenchymal stem cells into peripheral blood by the action of the fragment peptides of the nuclear proteins, and exhibition of the inflammation-suppressing effect and tissue regeneration effect by the cells.

### Example 3

### Efficacy of Nuclear Protein-Derived Peptides on Psoriasis

### (1) Materials and Methods

### i) Drugs

To induce psoriasis by imiquimod, a cream containing 5% imiquimod (Beseruna cream 5%, Mochida Pharmaceutical Co., Ltd.) was used. In the drawing corresponding to the Example of the present application, imiquimod is referred to as IMQ. In addition, the peptide NP-3 (TFA salt) described in Example 1 was used as a test substance.

### ii) Generation of Psoriasis Model Mice

C57BL/6 mice (7 weeks old, female, body weight about 20 g) were prepared. To induce psoriasis, a cream containing 5% imiquimod was applied on the auricular skin of the mice at an amount of 25 mg/ear/day (1.25 mg/ear/day imiquimod) once a day for seven days. Moreover, mice without imiquimod application (hereinafter, referred to as “normal mice”) were used as a comparison target.

iii) Peptide Administration

The psoriasis model mice prepared as described above were divided into a peptide-administered group (n=4) and a control group (n=4). Administration of the test substance was carried out from the first day of the start of imiquimod application (Day 1) for seven days, by injecting into the tail vein 100 μL/day of the NP-3 peptide solution which has been adjusted to a concentration of 1 μg/μL using physiological saline as a solvent (5 mg/kg/day as peptide dose). In the control group, physiological saline was injected into the tail vein in an amount of 100 μL/day, from the first day of the start of imiquimod application for seven days. No substance was administered to normal mice (n=4).

### iv) Evaluation of the Effects of Peptide Administration

During the test period, the auricle thickness of mice was measured daily using a micrometer (manufactured by Mitutoyo Co., Ltd., Product No. CLM1-15QM), and the size of change from the auricle thickness before the start of imiquimod application (Day 0) was calculated. The degree of skin thickening was evaluated using the size of change as an index.

### (2) Result

Changes in auricular thickness of mice during the test period are shown in FIG. 9 (see “Non treat” for normal mice, “IMQ/saline” for the control group, and “IMQ/NP-3” for the peptide NP-3 administration group). The auricular thickness of psoriasis model mice (the control group and peptide NP-3 administration group) increased with the passage of days. In the peptide NP-3 administration group, the increase in auricular thickness was suppressed as compared with the control group.

Symptoms of psoriasis vulgaris include erythema, thickening, scales/desquamation of the skin, which are caused by abnormalities in the immune system and the resulting inflammation, and hyperproliferation of keratinocytes. The mouse model used in this experiment is a model that causes psoriasis vulgaris-like symptoms (erythema and thickening) by applying imiquimod to the skin of the ear. As a result of the experiment, imiquimod-induced skin thickening was suppressed by administration of fragment peptide of the nuclear protein of the present application. This is thought to be the result of mobilization of mesenchymal stem cells into peripheral blood by the effect of the fragment peptide of the nuclear protein, and exhibition of the immune modulatory effect and inflammation-suppressing effect by the cells.

### INDUSTRIAL APPLICABILITY

The nuclear proteins or fragment peptides thereof of the present application can be used as therapeutic agents for inflammatory diseases, autoimmune diseases, fibrotic diseases, and diseases accompanied by tissue damage/ischemia/necrosis.

We claim:

1. A method for treating a disease or pathological condition in a subject by mobilizing mesenchymal stem cells to peripheral blood, which comprises administering to the subject an effective amount of a nuclear protein or a fragment peptide thereof.

2. The method of claim 1, wherein the treatment of a disease or pathological condition is selected from inflammation-suppressing therapy, immunomodulatory therapy, tissue regeneration-inducing therapy, and tissue fibrosis-suppressing therapy.

3. The method of claim 1, wherein the disease or pathological condition is selected from an inflammatory disease, an autoimmune disease, a disease accompanied by tissue damage, ischemia, or necrosis, and a fibrotic disease.

4. The method of claim 1, wherein the disease or pathological condition is selected from inflammatory bowel disease and psoriasis.

5. A method for treating a disease selected from inflammatory bowel disease or psoriasis in a subject, which comprises administering to the subject an effective amount of a nuclear protein or a fragment peptide thereof.

6. The method of claim 1, wherein the nuclear protein or fragment peptide thereof comprises a nuclear localization signal.

7. The method of claim 1, wherein the nuclear protein or fragment peptide thereof is a nuclear protein involved in transcription regulation or a fragment peptide thereof.

8. The method of claim 1, wherein the nuclear protein or fragment peptide thereof is a nuclear protein selected from the following or a fragment peptide thereof:
(1) BTF3 protein;
(2) SUPT16H protein;
(3) YBX1 protein;
(4) NPM1 protein;
(5) PA2G4 protein;
(6) PFDN5 protein;
(7) PSMC3 protein;
(8) HNRNPK protein; and
(9) a nuclear protein functionally equivalent to a protein selected from (1) to (8).

9. The method of claim 1, wherein the nuclear protein or fragment peptide thereof is a nuclear protein selected from the following or a fragment peptide thereof:
(a) a nuclear protein comprising an amino acid sequence selected from SEQ ID NOs: 1 to 34;
(b) a nuclear protein comprising an amino acid sequence resulting from substitution, deletion, insertion, or addition of one or more amino acids in an amino acid sequence selected from SEQ ID NOs: 1 to 34; and
(c) a nuclear protein comprising an amino acid sequence with a sequence identity of about 80% or higher with an amino acid sequence selected from SEQ ID NOs: 1 to 34.

10. The method of claim 1, wherein the fragment peptide of the nuclear protein is a fragment peptide selected from the following:
(a) a nuclear protein fragment peptide consisting of a portion of an amino acid sequence selected from SEQ ID NOs: 1 to 34;
(b) a nuclear protein fragment peptide comprising an amino acid sequence selected from SEQ ID NOs: 35 to 56;
(c) a nuclear protein fragment peptide consisting of a portion of an amino acid sequence selected from SEQ ID NOs: 35 to 56;
(d) a nuclear protein fragment peptide comprising an amino acid sequence resulting from substitution, deletion, insertion, or addition of one or more amino acids in an amino acid sequence selected from SEQ ID NOs: 35 to 56; and
(e) a nuclear protein fragment peptide comprising an amino acid sequence with a sequence identity of about 80% or higher with an amino acid sequence selected from SEQ ID NOs: 35 to 56.

11. A fragment peptide of a nuclear protein selected from the following:
(1) BTF3 protein;
(2) SUPT16H protein;
(3) YBX1 protein;
(4) NPM1 protein;
(5) PA2G4 protein;
(6) PFDN5 protein;
(7) PSMC3 protein;
(8) HNRNPK protein; and
(9) a nuclear protein functionally equivalent to a protein selected from (1) to (8).

12. The fragment peptide of claim 11, comprising a nuclear localization signal.

13. The fragment peptide of claim 11, which is a fragment peptide of a nuclear protein selected from the following:
(a) a nuclear protein comprising an amino acid sequence selected from SEQ ID NOs: 1 to 34;
(b) a nuclear protein comprising an amino acid sequence resulting from substitution, deletion, insertion, or addition of one or more amino acids in an amino acid sequence selected from SEQ ID NOs: 1 to 34; and
(c) a nuclear protein comprising an amino acid sequence with a sequence identity of about 80% or higher with an amino acid sequence selected from SEQ ID NOs: 1 to 34.

14. The fragment peptide of claim 11, which is selected from the following:
(a) a nuclear protein fragment peptide consisting of a portion of an amino acid sequence selected from SEQ ID NOs: 1 to 34;
(b) a nuclear protein fragment peptide comprising an amino acid sequence selected from SEQ ID NOs: 35 to 56;
(c) a nuclear protein fragment peptide consisting of a portion of an amino acid sequence selected from SEQ ID NOs: 35 to 56;
(d) a nuclear protein fragment peptide comprising an amino acid sequence resulting from substitution, deletion, insertion, or addition of one or more amino acids in an amino acid sequence selected from SEQ ID NOs: 35 to 56; and
(e) a nuclear protein fragment peptide comprising an amino acid sequence with a sequence identity of about 80% or higher with an amino acid sequence selected from SEQ ID NOs: 35 to 56.


SEQUENCE LISTING (130 sequences)

SEQ ID NO: 1 - protein, 204 aa, Mus musculus
MRRTGAPTQADSRGRGRARGGWPGAEATPSLPLGGSRGRESQMKETIMNQEKLAKLQAQVRIGGKGTARRKKKVVHRTATADDKKLQFSLKKLGVNNISGIEEVNMFTNQGTVIHFNNPKVQASLAANTFTITGHAETKQLTEMLPSILNQLGADSLTSLRRLAEALPKQSVDGKAPLATGEDDDDEVPDLVENFDEASKNEAN

SEQ ID NO: 2 - protein, 162 aa, Mus musculus
MKETIMNQEKLAKLQAQVRIGGKGTARRKKKVVHRTATADDKKLQFSLKKLGVNNISGIEEVNMFTNQGTVIHFNNPKVQASLAANTFTITGHAETKQLTEMLPSILNQLGADSLTSLRRLAEALPKQSVDGKAPLATGEDDDDEVPDLVENFDEASKNEAN

SEQ ID NO: 3 - protein, 1047 aa, Mus musculus
MAVTLDKDAYYRRVKRLYSNWRKGEDEYASIDAIVVSVGVDEEIVYAKSTALQTWLFGYELTDTIMVFCDDKIIFMASKKKVEFLKQIANTKGNENANGAPAITLLVREKNESNKSSFDKMIDAIKESKSGKKIGVFSKDKFPGEFMKSWSDCLNKEGFDKVDISAVVAYTIAVKEDGELNLMKKAASITSEVFNKFFKERVMEIVDADEKVRHSKLAESVEKAIEEKKYLAGADPSTVEMCYPPIIQSGGNYNLKFSVVSDKNHMHFGAITCAMGIRFKSYCSNLVRTLMVDPTQEVQENYNFLLQLQEELLKELRHGVKICDVYNSVMDVVKKQKPELLNKITKNLGFGMGIEFREGSLVINSKNQYKLKKGMVFSINLGFSDLTNKEGKKPEEKTYALFIGDTVLVDEDGPATILTSVKKKVKNVGIFLKNEDDEEEEEEKDEAEDLLGRGSRAALLTERTRNEMTAEEKRRAHQKELAAQLNEEAKRRLTEQKGEQQIQKARKSNVSYKNPSLMPKEPHIREMKIYIDKKYETVIMPVFGIATPFHIATIKNISMSVEGDYTYLRINFYCPGSALGRNEGNIFPNPEATFVKEITYRASNMKAPGEQTVPALNLQNAFRIIKEVQKRYKTREAEEKEKEGIVKQDSLVINLNRSNPKLKDLYIRPNIAQKRMQGSLEAHVNGFRFTSVRGDKVDILYNNIKHALFQPCDGEMIIVLHFHLKNAVMFGKKRHTDVQFYTEVGEITTDLGKHQHMHDRDDLYAEQMEREMRHKLKTAFKNFIEKVEALTKEELEFEVPFRDLGFNGAPYRSTCLLQPTSSALVNATEWPPFVVTLDEVELIHFERVQFHLKNFDMVIVYKDYSKKVTMINAIPVASLDPIKEWLNSCDLKYTEGVQSLNWTKIMKTIVDDPEGFFEQGGWSFLEPEGEGSDAEDGDSESEIEDETFNPSEDDYEEEEEDSDEDYSSEAEESDYSKESLGSEEESGKDWDELEEEARKADRESRYEEEEEQSRSMSRKRKASVHSSGRGSNRGSRHSSAPPKKKRK

SEQ ID NO: 4 - protein, 322 aa, Mus musculus
MSSEAETQQPPAAPAAALSAADTKPGSTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYARRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 5 - protein, 292 aa, Mus musculus
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEDEEDVKLLGMSGKRSAPGGGNKVPQKKVKLDEDDEDDDEDDEDDEDDDDDDFDEEETEEKVPVKKSVRDTPAKNAQKSNQNGKDLKPSTPRSKGQESFKKQEKTPKTPKGPSSVEDIKAKMQASIEKGGSLPKVEAKFINYVKNCFRMTDQEAIQDLWQWRKSL

SEQ ID NO: 6 - protein, 279 aa, Mus musculus
MEDSMDMDMSPLRPQNYLFVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEDEEDVKLLGMSGKRSAPGGGNKVPQKKVKLDEDDEDDDEDDEDDEDDDDDDFDEEETEEKVPVKKSVRDTPAKNAQKSNQNGKDLKPSTPRSKGQESFKKQEKTPKTPKGPSSVEDIKAKMQASIEKGGSLPKVEAKFINYVKNCFRMTDQEAIQDLWQWRKSL

SEQ ID NO: 7 - protein, 257 aa, Mus musculus
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEDEEDVKLLGMSGKRSAPGGGNKVPQKKVKLDEDDEDDDEDDEDDEDDDDDDFDEEETEEKVPVKKSVRDTPAKNAQKSNQNGKDLKPSTPRSKGQESFKKQEKTPKTPKGPSSVEDIKAKMQASIEKAH

SEQ ID NO: 8 - protein, 394 aa, Mus musculus
MSGEDEQQEQTIAEDLVVTKYKMGGDIANRVLRSLVEASSSGVSVLSLCEKGDAMIMEETGKIFKKEKEMKKGIAFPTSISVNNCVCHFSPLKSDQDYILKEGDLVKIDLGVHVDGFIANVAHTFVIGVAQGTQVTGRKADVIKAAHLCAEAALRLVKPGNQNTQVTEAWNKVAHSFNCTPIEGMLSHQLKQHVIDGEKTIIQNPTDQQKKDHEKAEFEVHEVYAVDVLVSSGEGKAKDAGQRTTIYKRDPSKQYGLKMKTSRAFFSEVERRFDAMPFTLRAFEDEKKARMGVVECAKHELLQPFNVLYEKEGEFVAQFKFTVLLMPNGPMRITSGPFEPDLYKSEMEVQDAELKALLQSSASRKTQKKKKKKASKTVENATSGETLEENGAGD

SEQ ID NO: 9 - protein, 154 aa, Mus musculus
MAQSINITELNLPQLEMLKNQLDQEVEFLSTSIAQLKVVQTKYVEAKDCLNVLNKSNEGKELLVPLTSSMYVPGKLHDVEHVLIDVGTGYYVEKTAEDAKDFFKRKIDFLTKQMEKIQPALQEKHAMKQAVMEMMSQKIQQLTALGAAQATVKA

SEQ ID NO: 10 - protein, 442 aa, Mus musculus
MQEMNLLPTPESPVTRQEKMATVWDEAEQDGIGEEVLKMSTEEIVQRTRLLDSEIKIMKSEVLRVTHELQAMKDKIKENSEKIKVNKTLPYLVSNVIELLDVDPNDQEEDGANIDLDSQRKGKCAVIKTSTRQTYFLPVIGLVDAEKLKPGDLVGVNKDSYLILETLPTEYDSRVKAMEVDERPTEQYSDIGGLDKQIQELVEAIVLPMNHKEKFENLGIQPPKGVLMYGPPGTGKTLLARACAAQTKATFLKLAGPQLVQMFIGDGAKLVRDAFALAKEKAPSIIFIDELDAIGTKRFDSEKAGDREVQRTMLELLNQLDGFQPNTQVKVIAATNRVDILDPALLRSGRLDRKIEFPMPNEEARARIMQIHSRKMNVSPDVNYEELARCTDDFNGAQCKAVCVEAGMIALRRGATELTHEDYMEGILEVQAKKKANLQYYA

SEQ ID NO: 11 - protein, 464 aa, Mus musculus
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYADVEGF

SEQ ID NO: 12 - protein, 463 aa, Mus musculus
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 13 - protein, 440 aa, Mus musculus
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYADVEGF

SEQ ID NO: 14 - protein, 439 aa, Mus musculus
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYDYSYAGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 15 - protein, 459 aa, Mus musculus
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYGGRGSYGDLGGPIITTQVTIPKDLAGSIIGKGGQRIKQIRHESGASIKIDEPLEGSEDRIITITGTQDQIQNAQYLLQNSVKQYADVEGF

SEQ ID NO: 16 - protein, 458 aa, Mus musculus
METEQPEETFPNTETNGEFGKRPAEDMEEEQAFKRSRNTDEMVELRILLQSKNAGAVIGKGGKNIKALRTDYNASVSVPDSSGPERILSISADIETIGEILKKIIPTLEEGLQLPSPTATSQLPLESDAVECLNYQHYKGSDFDCELRLLIHQSLAGGIIGVKGAKIKELRENTQTTIKLFQECCPHSTDRVVLIGGKPDRVVECIKIILDLISESPIKGRAQPYDPNFYDETYDYGGFTMMFDDRRGRPVGFPMRGRGGFDRMPPGRGGRPMPPSRRDYDDMSPRRGPPPPPPGRGGRGGSRARNLPLPPPPPPRGGDLMAYDRRGRPGDRYDGMVGFSADETWDSAIDTWSPSEWQMAYEPQGGSGYGGRGSYGDLGGPIITTQVTIPKDLAGSIIGKGGQRIKQIRHESGASIKIDEPLEGSEDRIITITGTQDQIQNAQYLLQNSVKQYSGKFF

SEQ ID NO: 17 - protein, 206 aa, Homo sapiens
MRRTGAPAQADSRGRGRARGGCPGGEATLSQPPPRGGTRGQEPQMKETIMNQEKLAKLQAQVRIGGKGTARRKKKVVHRTATADDKKLQFSLKKLGVNNISGIEEVNMFTNQGTVIHFNNPKVQASLAANTFTITGHAETKQLTEMLPSILNQLGADSLTSLRRLAEALPKQSVDGKAPLATGEDDDDEVPDLVENFDEASKNEAN

SEQ ID NO: 18 - protein, 162 aa, Homo sapiens; identical to SEQ ID NO: 2

SEQ ID NO: 19 - protein, 1047 aa, Homo sapiens
MAVTLDKDAYYRRVKRLYSNWRKGEDEYANVDAIVVSVGVDEEIVYAKSTALQTWLFGYELTDTIMVFCDDKIIFMASKKKVEFLKQIANTKGNENANGAPAITLLIREKNESNKSSFDKMIEAIKESKNGKKIGVFSKDKFPGEFMKSWNDCLNKEGFDKIDISAVVAYTIAVKEDGELNLMKKAASITSEVFNKFFKERVMEIVDADEKVRHSKLAESVEKAIEEKKYLAGADPSTVEMCYPPIIQSGGNYNLKFSVVSDKNHMHFGAITCAMGIRFKSYCSNLVRTLMVDPSQEVQENYNFLLQLQEELLKELRHGVKICDVYNAVMDVVKKQKPELLNKITKNLGFGMGIEFREGSLVINSKNQYKLKKGMVFSINLGFSDLTNKEGKKPEEKTYALFIGDTVLVDEDGPATVLTSVKKKVKNVGIFLKNEDEEEEEEEKDEAEDLLGRGSRAALLTERTRNEMTAEEKRRAHQKELAAQLNEEAKRRLTEQKGEQQIQKARKSNVSYKNPSLMPKEPHIREMKIYIDKKYETVIMPVFGIATPFHIATIKNISMSVEGDYTYLRINFYCPGSALGRNEGNIFPNPEATFVKEITYRASNIKAPGEQTVPALNLQNAFRIIKEVQKRYKTREAEEKEKEGIVKQDSLVINLNRSNPKLKDLYIRPNIAQKRMQGSLEAHVNGFRFTSVRGDKVDILYNNIKHALFQPCDGEMIIVLHFHLKNAIMFGKKRHTDVQFYTEVGEITTDLGKHQHMHDRDDLYAEQMEREMRHKLKTAFKNFIEKVEALTKEELEFEVPFRDLGFNGAPYRSTCLLQPTSSALVNATEWPPFVVTLDEVELIHFERVQFHLKNFDMVIVYKDYSKKVTMINAIPVASLDPIKEWLNSCDLKYTEGVQSLNWTKIMKTIVDDPEGFFEQGGWSFLEPEGEGSDAEEGDSESEIEDETFNPSEDDYEEEEEDSDEDYSSEAEESDYSKESLGSEEESGKDWDELEEEARKADRESRYEEEEEQSRSMSRKRKASVHSSGRGSNRGSRHSSAPPKKKRK

SEQ ID NO: 20 - protein, 324 aa, Homo sapiens
MSSEAETQQPPAAPPAAPALSAADTKPGTTGSGAGSGGPGGLTSAAPAGGDKKVIATKVLGTVKWFNVRNGYGFINRNDTKEDVFVHQTAIKKNNPRKYLRSVGDGETVEFDVVEGEKGAEAANVTGPGGVPVQGSKYAADRNHYRRYPRRRGPPRNYQQNYQNSESGEKNEGSESAPEGQAQQRRPYRRRRFPPYYMRRPYGRRPQYSNPPVQGEVMEGADNQGAGEQGRPVRQNMYRGYRPRFRRGPPRQRQPREDGNEEDKENQGDETQGQQPPQRRYRRNFNYRRRRPENPKPQDGKETKAADPPAENSSAPEAEQGGAE

SEQ ID NO: 21 - protein, 294 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 22 - protein, 265 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKGQESFKKQEKTPKTPKGPSSVEDIKAKMQASIEKGGSLPKVEAKFINYVKNCFRMTDQEAIQDLWQWRKSL

SEQ ID NO: 23 - protein, 259 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKAH

SEQ ID NO: 24 - protein, 230 aa, Homo sapiens
MNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKSIRDTPAKNAQKSNQNGKDSKPSSTPRSKGQESFKKQEKTPKTPKGPSSVEDIKAKMQASIEKGGSLPKVEAKFINYVKNCFRMTDQEAIQDLWQWRKSL

SEQ ID NO: 25 - protein, 230 aa, Homo sapiens
MEDSMDMDMSPLRPQNYLFGCELKADKDYHFKVDNDENEHQLSLRTVSLGAGAKDELHIVEAEAMNYEGSPIKVTLATLKMSVQPTVSLGGFEITPPVVLRLKCGSGPVHISGQHLVAVEEDAESEDEEEEDVKLLSISGKRSAPGGGSKVPQKKVKLAADEDDDDDDEEDDDEDDDDDDFDDEEAEEKAPVKKGQESFKKQEKTPKTPKGPSSVEDIKAKMQASIEKAH

SEQ ID NO: 26 - protein, 167 aa, Homo sapiens
MEDSMDMDMSPLRPQNYLFAVEEDAESEDEEEEDVKLLSISGKRSAPGGGSKVPQKKVKLAADEDDDDDDEEDDDEDDDDDDFDDEEAEEKAPVKKGQESFKKQEKTPKTPKGPSSVEDIKAKMQASIEKGGSLPKVEAKFINYVKNCFRMTDQEAIQDLWQWRKSL

SEQ ID NO: 27 - protein, 394 aa, Homo sapiens
MSGEDEQQEQTIAEDLVVTKYKMGGDIANRVLRSLVEASSSGVSVLSLCEKGDAMIMEETGKIFKKEKEMKKGIAFPTSISVNNCVCHFSPLKSDQDYILKEGDLVKIDLGVHVDGFIANVAHTFVVDVAQGTQVTGRKADVIKAAHLCAEAALRLVKPGNQNTQVTEAWNKVAHSFNCTPIEGMLSHQLKQHVIDGEKTIIQNPTDQQKKDHEKAEFEVHEVYAVDVLVSSGEGKAKDAGQRTTIYKRDPSKQYGLKMKTSRAFFSEVERRFDAMPFTLRAFEDEKKARMGVVECAKHELLQPFNVLYEKEGEFVAQFKFTVLLMPNGPMRITSGPFEPDLYKSEMEVQDAELKALLQSSASRKTQKKKKKKASKTAENATSGETLEENEAGD

SEQ ID NO: 28 - protein, 154 aa, Homo sapiens
MAQSINITELNLPQLEMLKNQLDQEVEFLSTSIAQLKVVQTKYVEAKDCLNVLNKSNEGKELLVPLTSSMYVPGKLHDVEHVLIDVGTGYYVEKTAEDAKDFFKRKIDFLTKQMEKIQPALQEKHAMKQAVMEMMSQKIQQLTALGAAQATAKA

SEQ ID NO: 29 - protein, 109 aa, Homo sapiens
MAQSINITELNLPQLEMLKNQLDQMYVPGKLHDVEHVLIDVGTGYYVEKTAEDAKDFFKRKIDFLTKQMEKIQPALQEKHAMKQAVMEMMSQKIQQLTALGAAQATAKA

SEQ ID NO: 30 - protein, 439 aa, Homo sapiens
MNLLPNIESPVTRQEKMATVWDEAEQDGIGEEVLKMSTEEIIQRTRLLDSEIKIMKSEVLRVTHELQAMKDKIKENSEKIKVNKTLPYLVSNVIELLDVDPNDQEEDGANIDLDSQRKGKCAVIKTSTRQTYFLPVIGLVDAEKLKPGDLVGVNKDSYLILETLPTEYDSRVKAMEVDERPTEQYSDIGGLDKQIQELVEAIVLPMNHKEKFENLGIQPPKGVLMYGPPGTGKTLLARACAAQTKATFLKLAGPQLVQMFIGDGAKLVRDAFALAKEKAPSIIFIDELDAIGTKRFDSEKAGDREVQRTMLELLNQLDGFQPNTQVKVIAATNRVDILDPALLRSGRLDRKIEFPMPNEEARARIMQIHSRKMNVSPDVNYEELARCTDDFNGAQCKAVCVEAGMIALRRGATELTHEDYMEGILEVQAKKKANLQYYA

SEQ ID NO: 31 - protein, 464 aa, Homo sapiens; identical to SEQ ID NO: 11

SEQ ID NO: 32 - protein, 463 aa, Homo sapiens; identical to SEQ ID NO: 12

SEQ ID NO: 33 - protein, 439 aa, Homo sapiens; identical to SEQ ID NO: 14

SEQ ID NO: 34 - protein, 440 aa, Homo sapiens; identical to SEQ ID NO: 13

SEQ ID NO: 35 - protein, 30 aa, Mus musculus
GGKGTARRKKKVVHRTATADDKKLQFSLKK

SEQ ID NO: 36 - protein, 30 aa, Mus musculus
REKNESNKSSFDKMIDAIKESKSGKKIGVF

SEQ ID NO: 37 - protein, 30 aa, Mus musculus
SAPEGQAQQRRPYRRRRFPPYYMRRPYARR

SEQ ID NO: 38 - protein, 30 aa, Mus musculus
GEQGRPVRQNMYRGYRPRFRRGPPRQRQPR

SEQ ID NO: 39 - protein, 30 aa, Mus musculus
QPPQRRYRRNFNYRRRRPENPKPQDGKETK

SEQ ID NO: 40 - protein, 30 aa, Mus musculus
NGKDLKPSTPRSKGQESFKKQEKTPKTPKG

SEQ ID NO: 41 - protein, 30 aa, Mus musculus
KGQESFKKQEKTPKTPKGPSSVEDIKAKMQ

SEQ ID NO: 42 - protein, 30 aa, Mus musculus
VSVLSLCEKGDAMIMEETGKIFKKEKEMKK

SEQ ID NO: 43 - protein, 30 aa, Mus musculus
AKDFFKRKIDFLTKQMEKIQPALQEKHAMK

SEQ ID NO: 44 - protein, 30 aa, Mus musculus
IKIMKSEVLRVTHELQAMKDKIKENSEKIK

SEQ ID NO: 45 - protein, 30 aa, Mus musculus
MFDDRRGRPVGFPMRGRGGFDRMPPGRGGR

SEQ ID NO: 46 - protein, 30 aa, Homo sapiens; identical to SEQ ID NO: 35

SEQ ID NO: 47 - protein, 30 aa, Homo sapiens
REKNESNKSSFDKMIEAIKESKNGKKIGVF

SEQ ID NO: 48 - protein, 30 aa, Homo sapiens
SAPEGQAQQRRPYRRRRFPPYYMRRPYGRR

SEQ ID NO: 49 - protein, 30 aa, Homo sapiens; identical to SEQ ID NO: 38

SEQ ID NO: 50 - protein, 30 aa, Homo sapiens; identical to SEQ ID NO: 39

SEQ ID NO: 51 - protein, 31 aa, Homo sapiens
NGKDSKPSSTPRSKGQESFKKQEKTPKTPKG

SEQ ID NO: 52 - protein, 30 aa, Homo sapiens; identical to SEQ ID NO: 41

SEQ ID NO: 53 - protein, 30 aa, Homo sapiens; identical to SEQ ID NO: 42

SEQ ID NO: 54 - protein, 30 aa, Homo sapiens; identical to SEQ ID NO: 43

SEQ ID NO: 55 - protein, 30 aa, Homo sapiens; identical to SEQ ID NO: 44

SEQ ID NO: 56 - protein, 30 aa, Homo sapiens; identical to SEQ ID NO: 45

SEQ ID NO: 57 - DNA, 615 bp, Mus musculus
  source 1..615: mol_type = genomic DNA
atgcgacggacgggcgctcccacccaggcggactctcgggggcgagggcgagccaggggcggctggcccggggccgaagcgacgccctctctccctctcggcggaagccgaggacgggagtctcagatgaaagaaacaatcatgaaccaggagaaactcgccaaactgcaggcacaagtgcgcattggtgggaaaggaactgctcgtaggaagaagaaggtggttcacagaacggccacagcagacgataagaaactgcagttctccttaaagaagttaggggtgaacaacatctctggtattgaagaggtgaacatgtttacaaaccaaggaacagtgatccattttaacaaccctaaagttcaggcatccctggcagcaaacaccttcaccattacaggccacgctgagacaaagcagctgacagaaatgcttcccagcatcctcaaccagcttggtgcagacagcctgactagtttaaggagactggctgaagctctgcccaaacaatctgtggatggaaaagcaccccttgctactggagaggatgatgatgatgaagttccagatctggtggagaattttgatgaggcttctaagaatgaggcaaactga

SEQ ID NO: 58 - DNA, 489 bp, Mus musculus
  source 1..489: mol_type = genomic DNA
atgaaagaaacaatcatgaaccaggagaaactcgccaaactgcaggcacaagtgcgcattggtgggaaaggaactgctcgtaggaagaagaaggtggttcacagaacggccacagcagacgataagaaactgcagttctccttaaagaagttaggggtgaacaacatctctggtattgaagaggtgaacatgtttacaaaccaaggaacagtgatccattttaacaaccctaaagttcaggcatccctggcagcaaacaccttcaccattacaggccacgctgagacaaagcagctgacagaaatgcttcccagcatcctcaaccagcttggtgcagacagcctgactagtttaaggagactggctgaagctctgcccaaacaatctgtggatggaaaagcaccccttgctactggagaggatgatgatgatgaagttccagatctggtggagaattttgatgaggcttctaagaatgaggcaaactga

SEQ ID NO: 59 - DNA, 3144 bp, Mus musculus
  source 1..3144: mol_type = genomic DNA
atggctgtgactctggacaaagacgcgtactaccggcgagtgaagagattgtacagtaactggcggaaaggagaagatgagtatgccagtattgatgccattgttgtatcggtgggtgttgatgaagaaattgtgtatgccaagtcaactgccttacagacgtggctctttggttatgaactaactgatacaatcatggtcttctgtgatgacaaaatcatcttcatggccagcaaaaagaaggtggagtttctgaaacagattgccaatactaaaggcaatgagaatgctaatggagcccctgccatcacactgcttgtcagagagaagaatgaaagtaacaagagcagctttgacaaaatgattgacgctatcaaagaaagcaagagcggcaagaagatcggagtgttcagcaaagacaagttccctggagagttcatgaagagctggagtgattgtctcaacaaggagggctttgacaaagtagacatcagcgctgttgtggcatacaccattgctgtgaaagaggacggtgagctcaacctgatgaagaaagcagccagcatcacctctgaggtcttcaacaagttctttaaggaaagagtcatggaaatagtggatgcagatgagaaagttcggcatagcaaattggctgagtctgtggaaaaggccattgaagaaaaaaaatacctagctggggcagatccttctactgtggaaatgtgttaccctcctatcattcagagtggtggcaactataatctcaagttcagtgtggtgagtgataagaatcatatgcattttggggccattacgtgtgccatgggcattcgctttaaatcttactgctccaaccttgttcgcactctgatggttgaccccactcaggaagttcaagaaaattacaactttttacttcaacttcaagaggagttgctaaaggaattaagacatggtgtgaagatatgtgatgtgtataactctgtcatggatgtggttaagaagcagaaaccagaattgctgaacaaaattacaaagaatctaggatttgggatgggaattgaattccgtgaaggctctctagtaatcaatagtaaaaatcagtacaagctaaagaaagggatggtttttagcatcaatctaggattttcagacctgactaacaaagaagggaaaaagccagaagagaaaacctatgccctgttcattggtgacacggtgcttgtagatgaggatggtccagccactattcttacttctgtgaaaaagaaagtaaagaatgtggggattttcctgaagaatgaggatgatgaagaggaggaggaagagaaagatgaggcagaggaccttttgggaagaggctctagggcggcattacttactgaaagaacaaggaatgagatgactgcagaagagaagcgaagagcacatcagaaggaactggcagcacagctcaacgaggaagcgaagaggaggctgacagagcagaaaggggaacagcagattcagaaagctcgaaaatctaatgtgtcctataaaaacccatctctgatgcctaaggaaccacatattcgggagatgaagatctatattgataagaaatatgagactgtgataatgcctgtgtttggcattgccacacccttccatattgccacaatcaagaacataagtatgtctgtcgaaggagactatacttacttgcgaatcaacttctattgtccaggcagtgctctgggcaggaatgagggcaacatctttcctaaccctgaagccacttttgtcaaggaaattacataccgagcttcaaatatgaaagcacctggagagcagactgtacccgccttaaatcttcagaatgcgttccgaattataaaagaagtacaaaaacgttacaagaccagagaagctgaagagaaagaaaaagagggcattgtaaaacaagactcgctggtgatcaacctaaaccggagtaatccaaaactgaaagacctgtacattcgtccaaacattgcccagaagagaatgcagggctcactggaggctcatgtcaatggtttccgattcacatctgttcgaggagacaaggtggatattctgtacaataatatcaagcatgcgctgttccagccctgtgatggcgagatgattattgttttgcactttcacctcaagaatgctatcatgtttgggaagaagcgacacacagatgtacagttctacacagaagttggagagatcactacagatttggggaaacatcaacacatgcatgaccgagatgacctgtatgctgagcagatggaacgagaaatgagacacaaactgaaaacggcctttaaaaatttcattgaaaaagtagaggccctaacaaaggaggagctggagtttgaagtgccatttagggacctggggtttaatggggctccctacaggagtacctgcctccttcagcccactagtagtgccctggtaaatgctacagagtggccaccctttgtggtgacactggatgaagtggagctgatccattttgagagggtccagtttcacctgaagaactttgatatggtcattgtctacaaggattacagcaagaaagtcacgatgatcaatgctattcctgttgcctctctagaccccatcaaggagtggctgaattcctgtgacctaaagtacacagaaggagttcagtctctcaactggactaaaatcatgaagaccattgttgatgaccccgagggcttctttgaacaaggtggctggtccttcctggaacctgagggtgaggggagtgacgctgaggacggagactcggagtctgaaattgaagatgagactttcaatccttctgaagatgactatgaagaggaagaagaggacagcgatgaagattactcatcagaagctgaagaatcagactattctaaggaatctctgggaagtgaagaagaaagcgggaaggactgggatgagctagaagaagaagcacgaaaagcggaccgggaaagccgttatgaggaggaagaagaacagagccgaagtatgagccggaagaggaaggcatctgtacatagttcaggccgtggctctaaccgtggttccagacacagctctgcacccccgaagaaaaagagaaagtaa

SEQ ID NO: 60 - DNA, 969 bp, Mus musculus
  source 1..969: mol_type = genomic DNA
atgagcagcgaggccgagacccagcagccgcccgccgcccccgccgccgccctcagcgccgccgacaccaagcccggctccacgggcagcggcgcgggtagtggcggcccgggtggcctcacatcggcggcgcccgccggcggggacaagaaggtcatcgcaacgaaggttttgggaacagtcaaatggttcaatgtaaggaacggatacggtttcatcaacaggaatgacaccaaggaagacgtatttgtacaccagactgccataaagaagaataaccccaggaagtaccttcgcagtgtaggcgatggagagactgtggagtttgatgttgttgaaggagaaaagggtgcggaggcagcaaatgttacaggccctggtggagttccagttcaaggcagtaaatacgcagcagaccgtaaccattatagacgctatccacgtcgtaggggtcctccacgcaattaccagcaaaattaccagaatagtgagagtggggaaaagaacgagggatcggaaagcgctcctgaaggccaggcccaacaacgccggccctatcgcaggcgaaggttcccaccttactacatgcggagaccctatgcgcgtcgaccacagtattccaacccccctgtgcaaggagaagtgatggagggtgctgacaaccagggtgcaggagagcaaggtagaccagtgagacagaatatgtatcggggttacagaccacgattccgaaggggccctcctcgccaaagacagcctagagaggatggcaatgaagaggacaaagaaaatcaaggagatgagacccaaggtcagcagccacctcaacgtcggtatcgccgaaacttcaattaccgacgcagacgcccagagaaccctaaaccacaagatggcaaagagacaaaagcagccgatccaccagctgagaattcgtccgctcccgaggctgagcagggcggggctgagtaa

SEQ ID NO: 61 - DNA, 879 bp, Mus musculus
  source 1..879: mol_type = genomic DNA
atggaagactcgatggatatggacatgagtcctcttaggcctcagaactaccttttcggctgtgaactaaaggctgacaaagactatcactttaaagtggataatgatgaaaatgagcaccagttgtcattaagaacggtcagtttaggagcaggggcaaaagatgagttacacatcgtagaggcagaagcaatgaactatgaaggcagtccaattaaagtaacactggcaactttgaaaatgtctgtacaaccaacagtttccctagggggctttgaaattacaccacctgtggtcttacggttgaagtgtggttcagggcctgtgcacattagtggacagcatctagtagctgtagaggaagatgcagagtctgaagatgaagatgaggaggacgtaaaactcttaggcatgtctggaaagcgatctgctcctggaggtggtaacaaggttccacagaaaaaagtaaaacttgatgaagatgatgaggacgatgatgaggacgatgaggatgatgaggatgatgatgatgatgattttgatgaagaggaaactgaagaaaaggtcccagtgaagaaatctgtacgagataccccagccaaaaatgcacaaaaatcaaaccaaaatggaaaagacttaaaaccatcaacaccgagatcaaagggtcaagagtccttcaaaaaacaggaaaagactcctaaaacaccaaaaggacctagttctgtagaagacattaaggcaaaaatgcaagcaagtatagaaaaaggcggttctcttcccaaagtggaagccaagttcattaattatgtgaagaattgtttccggatgactgaccaggaggctattcaagatctctggcagtggaggaaatctctttaa

SEQ ID NO: 62 - DNA, 840 bp, Mus musculus
  source 1..840: mol_type = genomic DNA
atggaagactcgatggatatggacatgagtcctcttaggcctcagaactaccttttcgtggataatgatgaaaatgagcaccagttgtcattaagaacggtcagtttaggagcaggggcaaaagatgagttacacatcgtagaggcagaagcaatgaactatgaaggcagtccaattaaagtaacactggcaactttgaaaatgtctgtacaaccaacagtttccctagggggctttgaaattacaccacctgtggtcttacggttgaagtgtggttcagggcctgtgcacattagtggacagcatctagtagctgtagaggaagatgcagagtctgaagatgaagatgaggaggacgtaaaactcttaggcatgtctggaaagcgatctgctcctggaggtggtaacaaggttccacagaaaaaagtaaaacttgatgaagatgatgaggacgatgatgaggacgatgaggatgatgaggatgatgatgatgatgattttgatgaagaggaaactgaagaaaaggtcccagtgaagaaatctgtacgagataccccagccaaaaatgcacaaaaatcaaaccaaaatggaaaagacttaaaaccatcaacaccgagatcaaagggtcaagagtccttcaaaaaacaggaaaagactcctaaaacaccaaaaggacctagttctgtagaagacattaaggcaaaaatgcaagcaagtatagaaaaaggcggttctcttcccaaagtggaagccaagttcattaattatgtgaagaattgtttccggatgactgaccaggaggctattcaagatctctggcagtggaggaaatctctttaa

SEQ ID NO: 63 - DNA, 774 bp, Mus musculus
  source 1..774: mol_type = genomic DNA
atggaagactcgatggatatggacatgagtcctcttaggcctcagaactaccttttcggctgtgaactaaaggctgacaaagactatcactttaaagtggataatgatgaaaatgagcaccagttgtcattaagaacggtcagtttaggagcaggggcaaaagatgagttacacatcgtagaggcagaagcaatgaactatgaaggcagtccaattaaagtaacactggcaactttgaaaatgtctgtacaaccaacagtttccctagggggctttgaaattacaccacctgtggtcttacggttgaagtgtggttcagggcctgtgcacattagtggacagcatctagtagctgtagaggaagatgcagagtctgaagatgaagatgaggaggacgtaaaactcttaggcatgtctggaaagcgatctgctcctggaggtggtaacaaggttccacagaaaaaagtaaaacttgatgaagatgatgaggacgatgatgaggacgatgaggatgatgaggatgatgatgatgatgattttgatgaagaggaaactgaagaaaaggtcccagtgaagaaatctgtacgagataccccagccaaaaatgcacaaaaatcaaaccaaaatggaaaagacttaaaaccatcaacaccgagatcaaagggtcaagagtccttcaaaaaacaggaaaagactcctaaaacaccaaaaggacctagttctgtagaagacattaaggcaaaaatgcaagcaagtatagaaaaagcgcattga

SEQ ID NO: 64 - DNA, 1185 bp, Mus musculus
  source 1..1185: mol_type = genomic DNA
atgtcgggcgaagacgagcagcaggagcaaactatcgccgaggacctggtcgtgaccaagtataagatggggggcgacatcgccaaccgggtgcttcgatctttggtggaggcttccagctcaggtgtgtctgtgctgagcttgtgtgagaaaggtgatgccatgattatggaagagacagggaagatcttcaagaaggaaaaggagatgaagaaaggtattgcctttcctaccagcatttccgtaaataactgtgtgtgtcacttctcccctttgaagagtgaccaggactatatactcaaggaaggtgacttggtaaaaattgaccttggggttcatgtggatggcttcattgccaatgtggctcacacttttgtaattggtgtagctcaggggacccaggtaacagggcggaaagcagatgtcattaaggccgctcacctatgtgctgaagctgccttacgactggtcaaacctggaaaccagaacacacaagtgactgaagcatggaacaaggttgctcactcatttaactgcacaccaatagaaggtatgctgtcacaccaattgaagcagcatgtgattgatggagagaagacgattatccagaaccctacagaccagcagaagaaggaccatgaaaaggcagaatttgaggtgcatgaggtttatgctgtagatgtcctcgtcagctcaggagaaggcaaggccaaagatgcaggacagagaaccaccatctacaagcgagacccctctaaacaatatggcctgaaaatgaaaacttcacgtgcctttttcagtgaggtggaacggcgttttgatgccatgccgtttactttaagagcatttgaagatgagaagaaggctcgaatgggtgtggtagagtgtgccaaacatgagttactacagccatttaacgttctctatgagaaggagggtgaatttgttgcccagtttaaatttacagttctactcatgcccaacggccccatgcggataaccagtggtccctttgagcctgacctgtacaagtctgagatggaggttcaagatgcagagctgaaggctcttctccagagttctgcaagtagaaaaacccagaaaaagaagaaaaagaaggcctccaagactgtagagaatgccaccagtggagaaaccttagaagagaatggagctggggactga

SEQ ID NO: 65 - DNA, 465 bp, Mus musculus
  source 1..465: mol_type = genomic DNA
atggcgcagtcgattaacatcacggagctgaatctgccacaactggaaatgctcaagaaccagctggaccaggaagtggagtttttgtccacgtccattgctcagctcaaggtggtccagaccaagtacgtggaagccaaggactgtctgaacgtgctgaacaagagcaacgagggaaaagaattactggtcccactgacgagttctatgtacgtccccgggaagctacacgatgtggagcatgtgcttattgatgtgggaaccggctactacgtggagaagacagctgaggacgccaaggacttcttcaaaaggaagatagacttcctcaccaaacagatggagaaaatccagccagcgctgcaggagaagcatgccatgaagcaggctgtcatggaaatgatgagccagaagattcagcagctcacagccctgggggcagcgcaggccacggtcaaagcctga

SEQ ID NO: 66 - DNA, 1329 bp, Mus musculus
  source 1..1329: mol_type = genomic DNA
atgcaggaaatgaatctgctgccgacgcccgagagtccagtgactcggcaggagaagatggcgaccgtgtgggatgaagctgagcaagatggcattggggaggaggtgctcaagatgtccacggaagagattgtccagcgcacacggctgttagacagcgagatcaagatcatgaagagtgaagtattgcgagtcacccatgaactccaagccatgaaagacaaaatcaaagagaacagtgagaaaatcaaagtgaacaaaaccctgccgtaccttgtctccaatgtcatcgagttgctggacgttgaccccaatgaccaggaggaggatggtgccaacattgacctggactctcagaggaagggcaagtgtgcggtgatcaaaacttctacccgacagacatacttcctgccagtgattgggttggtggatgcagaaaagctgaagccaggagacctggtgggtgtgaacaaagactcctatctgatcctggagaccctgcccactgaatatgactctcgggtgaaggccatggaggtggacgagcggcccacggagcaatacagtgacatcgggggcctggacaagcagatccaggagctggtggaagccattgtcttgcctatgaaccacaaagagaagtttgagaacttgggtatccagcccccaaaaggagtgctgatgtatgggccgcctggaacagggaagactctgcttgcccgagcctgtgctgctcagaccaaggccaccttcttgaagctggcaggccctcagctggtacagatgtttattggagatggcgccaagctggtccgtgatgcttttgccctggccaaggagaaggcaccatctattattttcatagacgaattggatgccattggtaccaaacgcttcgacagtgaaaaggcaggagaccgagaggtgcagaggaccatgctggagctactgaaccagctggacggctttcagcccaacactcaagtgaaggtaattgcagccactaacagggtggacatcctggatccagccctgctgcgctcaggccgcctagaccgcaagattgagtttccaatgcccaacgaggaggccagagccagaatcatgcagatccactcacggaagatgaatgtcagtcctgatgtgaactatgaagagctggctcggtgcactgatgacttcaatggagcccagtgcaaggccgtgtgtgtggaggcgggtatgatcgcattgcgcaggggagccacggaactcactcatgaggactacatggagggcatcctggaggttcaggccaagaagaaagccaacctacaatactatgcctag

SEQ ID NO: 67 - DNA, 1395 bp, Mus musculus
  source 1..1395: mol_type = genomic DNA
atggagaccgaacagccagaagaaaccttccccaacaccgaaaccaatggtgaatttggtaaacgccctgcagaagatatggaagaggagcaagcctttaaaagatctagaaatactgatgagatggttgaattgcgcattttgcttcagagcaagaatgctggagcagtgattggaaaaggaggcaagaatattaaggctctccgtacagactacaatgccagtgtttcagtcccagacagcagtggccccgagcgcatactgagtatcagtgctgatattgagacgattggagaaattctgaagaaaatcatccctaccttggaagagggcctgcagttgccatcacccactgcaaccagccagctcccgctcgaatctgatgctgtggaatgcttaaattaccaacattataaaggaagtgactttgattgcgagttgagactgttgattcatcagagtctggcaggaggaataattggtgttaaaggtgctaaaatcaaagaacttcgagaaaacactcagacaacaatcaagcttttccaggagtgctgccctcactctactgacagagttgttcttattggaggaaaacctgatagggttgtagaatgcatcaagatcatccttgaccttatatctgagtctcccatcaaaggacgtgcacaaccttatgatcccaacttttatgatgagacctatgattatggtggttttacaatgatgtttgatgaccgccgaggacgacctgtgggattccccatgaggggaagaggtggttttgacagaatgcctcctggtcggggtgggcgtcccatgcctccttctagaagagattatgatgatatgagccctcgtcgaggacctccaccaccaccacctggtcgaggtggccggggtggcagcagagcccggaatctgcctcttcctcctccaccaccacccagagggggagatctaatggcttatgacagaagaggaaggcctggagaccgctatgatggcatggttgggttcagtgctgatgaaacttgggattctgcaattgacacatggagcccatcagaatggcaaatggcttatgaaccacagggtggttctggatatgactattcttatgcagggggccgtggctcatatggtgatcttggcggacctattatcactacacaagtaactattcccaaagatttggctggatctattattggcaaaggtggtcagcggattaaacaaattcgtcatgaatctggagcatcaatcaaaattgatgaacctttagaaggatctgaagatcggatcattaccattacaggaacacaggaccagatacagaacgcacagtatttgctgcagaacagtgtgaagcagtatgcagatgttgaaggattctaa

SEQ ID NO: 68 - DNA, 1392 bp, Mus musculus
  source 1..1392: mol_type = genomic DNA
atggagaccgaacagccagaagaaaccttccccaacaccgaaaccaatggtgaatttggtaaacgccctgcagaagatatggaagaggagcaagcctttaaaagatctagaaatactgatgagatggttgaattgcgcattttgcttcagagcaagaatgctggagcagtgattggaaaaggaggcaagaatattaaggctctccgtacagactacaatgccagtgtttcagtcccagacagcagtggccccgagcgcatactgagtatcagtgctgatattgagacgattggagaaattctgaagaaaatcatccctaccttggaagagggcctgcagttgccatcacccactgcaaccagccagctcccgctcgaatctgatgctgtggaatgcttaaattaccaacattataaaggaagtgactttgattgcgagttgagactgttgattcatcagagtctggcaggaggaataattggtgttaaaggtgctaaaatcaaagaacttcgagaaaacactcagacaacaatcaagcttttccaggagtgctgccctcactctactgacagagttgttcttattggaggaaaacctgatagggttgtagaatgcatcaagatcatccttgaccttatatctgagtctcccatcaaaggacgtgcacaaccttatgatcccaacttttatgatgagacctatgattatggtggttttacaatgatgtttgatgaccgccgaggacgacctgtgggattccccatgaggggaagaggtggttttgacagaatgcctcctggtcggggtgggcgtcccatgcctccttctagaagagattatgatgatatgagccctcgtcgaggacctccaccaccaccacctggtcgaggtggccggggtggcagcagagcccggaatctgcctcttcctcctccaccaccacccagagggggagatctaatggcttatgacagaagaggaaggcctggagaccgctatgatggcatggttgggttcagtgctgatgaaacttgggattctgcaattgacacatggagcccatcagaatggcaaatggcttatgaaccacagggtggttctggatatgactattcttatgcagggggccgtggctcatatggtgatcttggcggacctattatcactacacaagtaactattcccaaagatttggctggatctattattggcaaaggtggtcagcggattaaacaaattcgtcatgaatctggagcatcaatcaaaattgatgaacctttagaaggatctgaagatcggatcattaccattacaggaacacaggaccagatacagaacgcacagtatttgctgcagaacagtgtgaagcagtattctggaaagtttttctaa

SEQ ID NO: 69 - DNA, 1323 bp, Mus musculus
  source 1..1323: mol_type = genomic DNA
atggagaccgaacagccagaagaaaccttccccaacaccgaaaccaatggtgaatttggtaaacgccctgcagaagatatggaagaggagcaagcctttaaaagatctagaaatactgatgagatggttgaattgcgcattttgcttcagagcaagaatgctggagcagtgattggaaaaggaggcaagaatattaaggctctccgtacagactacaatgccagtgtttcagtcccagacagcagtggccccgagcgcatactgagtatcagtgctgatattgagacgattggagaaattctgaagaaaatcatccctaccttggaagagtaccaacattataaaggaagtgactttgattgcgagttgagactgttgattcatcagagtctggcaggaggaataattggtgttaaaggtgctaaaatcaaagaacttcgagaaaacactcagacaacaatcaagcttttccaggagtgctgccctcactctactgacagagttgttcttattggaggaaaacctgatagggttgtagaatgcatcaagatcatccttgaccttatatctgagtctcccatcaaaggacgtgcacaaccttatgatcccaacttttatgatgagacctatgattatggtggttttacaatgatgtttgatgaccgccgaggacgacctgtgggattccccatgaggggaagaggtggttttgacagaatgcctcctggtcggggtgggcgtcccatgcctccttctagaagagattatgatgatatgagccctcgtcgaggacctccaccaccaccacctggtcgaggtggccggggtggcagcagagcccggaatctgcctcttcctcctccaccaccacccagagggggagatctaatggcttatgacagaagaggaaggcctggagaccgctatgatggcatggttgggttcagtgctgatgaaacttgggattctgcaattgacacatggagcccatcagaatggcaaatggcttatgaaccacagggtggttctggatatgactattcttatgcagggggccgtggctcatatggtgatcttggcggacctattatcactacacaagtaactattcccaaagatttggctggatctattattggcaaaggtggtcagcggattaaacaaattcgtcatgaatctggagcatcaatcaaaattgatgaacctttagaaggatctgaagatcggatcattaccattacaggaacacaggaccagatacagaacgcacagtatttgctgcagaacagtgtgaagcagtatgcagatgttgaaggattctaa

SEQ ID NO: 70 - DNA, 1320 bp, Mus musculus
  source 1..1320: mol_type = genomic DNA
atggagaccgaacagccagaagaaaccttccccaacaccgaaaccaatggtgaatttggtaaacgccctgcagaagatatggaagaggagcaagcctttaaaagatctagaaatactgatgagatggttgaattgcgcattttgcttcagagcaagaatgctggagcagtgattggaaaaggaggcaagaatattaaggctctccgtacagactacaatgccagtgtttcagtcccagacagcagtggccccgagcgcatactgagtatcagtgctgatattgagacgattggagaaattctgaagaaaatcatccctaccttggaagagtaccaacattataaaggaagtgactttgattgcgagttgagactgttgattcatcagagtctggcaggaggaataattggtgttaaaggtgctaaaatcaaagaacttcgagaaaacactcagacaacaatcaagcttttccaggagtgctgccctcactctactgacagagttgttcttattggaggaaaacctgatagggttgtagaatgcatcaagatcatccttgaccttatatctgagtctcccatcaaaggacgtgcacaaccttatgatcccaacttttatgatgagacctatgattatggtggttttacaatgatgtttgatgaccgccgaggacgacctgtgggattccccatgaggggaagaggtggttttgacagaatgcctcctggtcggggtgggcgtcccatgcctccttctagaagagattatgatgatatgagccctcgtcgaggacctccaccaccaccacctggtcgaggtggccggggtggcagcagagcccggaatctgcctcttcctcctccaccaccacccagagggggagatctaatggcttatgacagaagaggaaggcctggagaccgctatgatggcatggttgggttcagtgctgatgaaacttgggattctgcaattgacacatggagcccatcagaatggcaaatggcttatgaaccacagggtggttctggatatgactattcttatgcagggggccgtggctcatatggtgatcttggcggacctattatcactacacaagtaactattcccaaagatttggctggatctattattggcaaaggtggtcagcggattaaacaaattcgtcatgaatctggagcatcaatcaaaattgatgaacctttagaaggatctgaagatcggatcattaccattacaggaacacaggaccagatacagaacgcacagtatttgctgcagaacagtgtgaagcagtattctggaaagtttttctaa

SEQ ID NO: 71 - DNA, 1380 bp, Mus musculus
  source 1..1380: mol_type = genomic DNA
atggagaccgaacagccagaagaaaccttccccaacaccgaaaccaatggtgaatttggtaaacgccctgcagaagatatggaagaggagcaagcctttaaaagatctagaaatactgatgagatggttgaattgcgcattttgcttcagagcaagaatgctggagcagtgattggaaaaggaggcaagaatattaaggctctccgtacagactacaatgccagtgtttcagtcccagacagcagtggccccgagcgcatactgagtatcagtgctgatattgagacgattggagaaattctgaagaaaatcatccctaccttggaagagggcctgcagttgccatcacccactgcaaccagccagctcccgctcgaatctgatgctgtggaatgcttaaattaccaacattataaaggaagtgactttgattgcgagttgagactgttgattcatcagagtctggcaggaggaataattggtgttaaaggtgctaaaatcaaagaacttcgagaaaacactcagacaacaatcaagcttttccaggagtgctgccctcactctactgacagagttgttcttattggaggaaaacctgatagggttgtagaatgcatcaagatcatccttgaccttatatctgagtctcccatcaaaggacgtgcacaaccttatgatcccaacttttatgatgagacctatgattatggtggttttacaatgatgtttgatgaccgccgaggacgacctgtgggattccccatgaggggaagaggtggttttgacagaatgcctcctggtcggggtgggcgtcccatgcctccttctagaagagattatgatgatatgagccctcgtcgaggacctccaccaccaccacctggtcgaggtggccggggtggcagcagagcccggaatctgcctcttcctcctccaccaccacccagagggggagatctaatggcttatgacagaagaggaaggcctggagaccgctatgatggcatggttgggttcagtgctgatgaaacttgggattctgcaattgacacatggagcccatcagaatggcaaatggcttatgaaccacagggtggttctggatatgggggccgtggctcatatggtgatcttggcggacctattatcactacacaagtaactattcccaaagatttggctggatctattattggcaaaggtggtcagcggattaaacaaattcgtcatgaatctggagcatcaatcaaaattgatgaacctttagaaggatctgaagatcggatcattaccattacaggaacacaggaccagatacagaacgcacagtatttgctgcagaacagtgtgaagcagtatgcagatgttgaaggattctaa

SEQ ID NO: 72 - DNA, 1377 bp, Mus musculus
  source 1..1377: mol_type = genomic DNA
atggagaccgaacagccagaagaaaccttccccaacaccgaaaccaatggtgaatttggtaaacgccctgcagaagatatggaagaggagcaagcctttaaaagatctagaaatactgatgagatggttgaattgcgcattttgcttcagagcaagaatgctggagcagtgattggaaaaggaggcaagaatattaaggctctccgtacagactacaatgccagtgtttcagtcccagacagcagtggccccgagcgcatactgagtatcagtgctgatattgagacgattggagaaattctgaagaaaatcatccctaccttggaagagggcctgcagttgccatcacccactgcaaccagccagctcccgctcgaatctgatgctgtggaatgcttaaattaccaacattataaaggaagtgactttgattgcgagttgagactgttgattcatcagagtctggcaggaggaataattggtgttaaaggtgctaaaatcaaagaacttcgagaaaacactcagacaacaatcaagcttttccaggagtgctgccctcactctactgacagagttgttcttattggaggaaaacctgatagggttgtagaatgcatcaagatcatccttgaccttatatctgagtctcccatcaaaggacgtgcacaaccttatgatcccaacttttatgatgagacctatgattatggtggttttacaatgatgtttgatgaccgccgaggacgacctgtgggattccccatgaggggaagaggtggttttgacagaatgcctcctggtcggggtgggcgtcccatgcctccttctagaagagattatgatgatatgagccctcgtcgaggacctccaccaccaccacctggtcgaggtggccggggtggcagcagagcccggaatctgcctcttcctcctccaccaccacccagagggggagatctaatggcttatgacagaagaggaaggcctggagaccgctatgatggcatggttgggttcagtgctgatgaaacttgggattctgcaattgacacatggagcccatcagaatggcaaatggcttatgaaccacagggtggttctggatatgggggccgtggctcatatggtgatcttggcggacctattatcactacacaagtaactattcccaaagatttggctggatctattattggcaaaggtggtcagcggattaaacaaattcgtcatgaatctggagcatcaatcaaaattgatgaacctttagaaggatctgaagatcggatcattaccattacaggaacacaggaccagatacagaacgcacagtatttgctgcagaacagtgtgaagcagtattctggaaagtttttctaa

SEQ ID NO: 73 - DNA, 621 bp, Homo sapiens
  source 1..621: mol_type = genomic DNA
atgcgacggacaggcgcacccgctcaggctgactctcgggggcgaggtcgagccaggggcggctgccctgggggcgaggcgacgctgtctcaacctccacctcgcggcggaacccgaggacaggagcctcagatgaaagaaacaatcatgaaccaggaaaaactcgccaaactgcaggcacaagtgcgcattggtgggaaaggaactgctcgcagaaagaagaaggtggttcatagaacagccacagcagatgacaaaaaacttcagttctccttaaagaagttaggggtaaacaatatctctggtattgaagaggtgaatatgtttacaaaccaaggaacagtgatccactttaacaaccctaaagttcaggcatctctggcagcgaacactttcaccattacaggccatgctgagacaaagcagctgacagaaatgctacccagcatcttaaaccagcttggtgcggatagtctgactagtttaaggagactggccgaagctctgcccaaacaatctgtggatggaaaagcaccacttgctactggagaggatgatgatgatgaagttccagatcttgtggagaattttgatgaggcttccaagaatgaggcaaactga

SEQ ID NO: 74 - DNA, 489 bp, Homo sapiens
  source 1..489: mol_type = genomic DNA
atgaaagaaacaatcatgaaccaggaaaaactcgccaaactgcaggcacaagtgcgcattggtgggaaaggaactgctcgcagaaagaagaaggtggttcatagaacagccacagcagatgacaaaaaacttcagttctccttaaagaagttaggggtaaacaatatctctggtattgaagaggtgaatatgtttacaaaccaaggaacagtgatccactttaacaaccctaaagttcaggcatctctggcagcgaacactttcaccattacaggccatgctgagacaaagcagctgacagaaatgctacccagcatcttaaaccagcttggtgcggatagtctgactagtttaaggagactggccgaagctctgcccaaacaatctgtggatggaaaagcaccacttgctactggagaggatgatgatgatgaagttccagatcttgtggagaattttgatgaggcttccaagaatgaggcaaactga

SEQ ID NO: 75 - DNA, 3144 bp, Homo sapiens
  source 1..3144: mol_type = genomic DNA
atggctgtgactctggacaaagacgcttattatcggcgagtgaagagactgtacagcaattggcggaaaggagaagatgagtatgccaacgttgatgccattgttgtatcagtgggtgttgatgaagaaattgtttatgccaaatcaactgccttacagacatggctctttggttatgaactaactgatactatcatggtcttttgtgatgacaaaatcatctttatggccagcaagaaaaaagtggagttcttgaaacagattgccaacactaagggcaatgagaatgctaatggagcccctgccatcacactgctaatacgagaaaagaatgaaagtaataagagtagctttgacaaaatgattgaagccattaaagaaagcaagaatggcaagaagattggagtgttcagcaaagacaaattccctggagagttcatgaagagctggaatgactgcctcaacaaagaaggctttgacaaaatagatatcagtgcagttgtggcatataccatcgctgtaaaggaggatggggagctcaacctaatgaagaaagcagccagcatcacttctgaagtcttcaacaaattcttcaaggaaagagtcatggaaatagttgatgcagatgagaaagttcgacacagcaaactggctgagtctgtggaaaaggccattgaagagaaaaaataccttgctggggcagacccttctactgtggaaatgtgttaccctcctatcattcagagtggtggcaactataatctcaagttcagtgtggtgagtgacaagaatcatatgcactttggggctatcacttgtgccatgggtattcgcttcaagtcttactgctccaaccttgttcgcactttgatggttgatccttctcaagaagttcaagaaaattataactttttgctccagcttcaagaggagctgctgaaggaattaagacatggtgtgaagatatgtgacgtgtataacgctgtcatggacgtggttaaaaagcagaagccagaactgctgaacaaaattaccaaaaacctagggtttgggatgggaattgaattccgtgaaggctccctagtaatcaatagcaaaaatcaatacaaactgaagaaaggaatggttttcagcatcaatttaggattctcagacctgactaacaaggaggggaaaaagccagaagagaaaacctatgccctgttcattggtgacacagtgcttgtggatgaggatggcccagctactgttctcacttctgtgaagaagaaagtgaagaatgtggggattttcctaaagaatgaagatgaggaagaagaggaggaggagaaagatgaggcagaggaccttttgggaagaggttctcgggcagcattacttacagaaagaacaagaaatgaaatgactgcagaagagaagcgaagagcacatcagaaagaactagcggctcaactcaatgaagaagcaaagaggcgattgactgaacaaaagggagaacagcagattcagaaagctcgcaagtctaatgtgtcctataaaaacccatctctgatgcctaaggaaccacatattcgggaaatgaagatctacatcgataagaaatatgagactgtaataatgcccgtgtttggcattgcaacaccgtttcacattgccacaatcaagaatataagtatgtccgtggaaggagattatacttacttgcgaatcaacttttattgcccaggcagtgctctgggcaggaatgaaggcaacatctttcctaaccctgaagcgacttttgtcaaggaaattacataccgagcatcaaatattaaggcacccggagaacagacagtaccagccttgaaccttcagaatgctttccgaattattaaagaagtacagaaacgttataaaactcgagaagctgaagagaaagagaaggaggggattgtaaaacaagactcactggtgatcaatctaaaccggagtaatccgaaactgaaagatctatacattcgcccaaatattgcccaaaagaggatgcaaggctcactggaggcccatgtcaatggcttccgcttcacatctgttcgaggagacaaagtggatattttgtacaataatattaagcatgctttgttccagccctgtgatggagaaatgattattgtcttgcactttcacctcaagaatgccatcatgtttgggaagaagcggcacacggatgtgcagttctacacagaagtgggagagataaccacggacttggggaaacatcagcatatgcatgaccgagatgacctctatgctgagcagatggaacgagaaatgaggcacaaactgaaaacagcctttaaaaatttcattgagaaagtagaggctctaactaaggaggaactggaatttgaagtgccttttagggacttgggatttaacggagctccctataggagtacctgcctccttcagcccactagtagtgcgctggtaaatgctacggaatggccaccttttgtggtgacattggatgaggtagagctgatccactttgagcgggtccagtttcacctgaagaactttgatatggtaatcgtctacaaggactacagcaagaaagtgaccatgatcaacgccattcctgtagcctctcttgaccccatcaaggaatggttgaattcctgcgacctgaaatacacagaaggagtacagtccctcaactggactaaaatcatgaagaccattgttgatgaccctgagggcttcttcgaacaaggtggctggtctttcctggagcctgagggtgaggggagtgatgctgaagaaggggattcagagtctgaaattgaagatgagacttttaatccttcagaagatgactatgaagaggaagaggaggacagtgatgaagattattcatcagaagcagaagagtcagactattctaaggagtcattgggtagtgaagaagagagtggaaaggattgggatgaactggaggaagaagcccgaaaagcggaccgagaaagtcgttacgaggaagaagaagaacaaagtcgaagtatgagccggaagaggaaggcatctgtgcacagttcgggccgtggctctaaccgtggttccagacacagctctgcaccccccaagaaaaagaggaagtaa

SEQ ID NO: 76 - DNA, 975 bp, Homo sapiens
  source 1..975: mol_type = genomic DNA
atgagcagcgaggccgagacccagcagccgcccgccgccccccccgccgcccccgccctcagcgccgccgacaccaagcccggcactacgggcagcggcgcagggagcggtggcccgggcggcctcacatcggcggcgcctgccggcggggacaagaaggtcatcgcaacgaaggttttgggaacagtaaaatggttcaatgtaaggaacggatatggtttcatcaacaggaatgacaccaaggaagatgtatttgtacaccagactgccataaagaagaataaccccaggaagtaccttcgcagtgtaggagatggagagactgtggagtttgatgttgttgaaggagaaaagggtgcggaggcagcaaatgttacaggtcctggtggtgttccagttcaaggcagtaaatatgcagcagaccgtaaccattatagacgctatccacgtcgtaggggtcctccacgcaattaccagcaaaattaccagaatagtgagagtggggaaaagaacgagggatcggagagtgctcccgaaggccaggcccaacaacgccggccctaccgcaggcgaaggttcccaccttactacatgcggagaccctatgggcgtcgaccacagtattccaaccctcctgtgcagggagaagtgatggagggtgctgacaaccagggtgcaggagaacaaggtagaccagtgaggcagaatatgtatcggggatatagaccacgattccgcaggggccctcctcgccaaagacagcctagagaggacggcaatgaagaagataaagaaaatcaaggagatgagacccaaggtcagcagccacctcaacgtcggtaccgccgcaacttcaattaccgacgcagacgcccagaaaaccctaaaccacaagatggcaaagagacaaaagcagccgatccaccagctgagaattcgtccgctcccgaggctgagcagggcggggctgagtaa

SEQ ID NO: 77 - DNA, 885 bp, Homo sapiens
  source 1..885: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaa

SEQ ID NO: 78 - DNA, 798 bp, Homo sapiens
  source 1..798: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaa

SEQ ID NO: 79 - DNA, 780 bp, Homo sapiens
  source 1..780: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaagcgcattga

SEQ ID NO: 80 - DNA, 693 bp, Homo sapiens
  source 1..693: mol_type = genomic DNA
atgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaatctatacgagatactccagccaaaaatgcacaaaagtcaaatcagaatggaaaagactcaaaaccatcatcaacaccaagatcaaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaa

SEQ ID NO: 81 - DNA, 693 bp, Homo sapiens
  source 1..693: mol_type = genomic DNA
atggaagattcgatggacatggacatgagccccctgaggccccagaactatcttttcggttgtgaactaaaggccgacaaagattatcactttaaggtggataatgatgaaaatgagcaccagttatctttaagaacggtcagtttaggggctggtgcaaaggatgagttgcacattgttgaagcagaggcaatgaattacgaaggcagtccaattaaagtaacactggcaactttgaaaatgtctgtacagccaacggtttcccttgggggctttgaaataacaccaccagtggtcttaaggttgaagtgtggttcagggccagtgcatattagtggacagcacttagtagctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaaggacaagaatccttcaagaaacaggaaaaaactcctaaaacaccaaaaggacctagttctgtagaagacattaaagcaaaaatgcaagcaagtatagaaaaagcgcattga

SEQ ID NO: 82 - DNA, 504 bp, Homo sapiens
  source 1..504: mol_type = genomic DNA
atggaagattcgatggacatggacatgagccccctgaggccccagaactatcttttcgctgtggaggaagatgcagagtcagaagatgaagaggaggaggatgtgaaactcttaagtatatctggaaagcggtctgcccctggaggtggtagcaaggttccacagaaaaaagtaaaacttgctgctgatgaagatgatgacgatgatgatgaagaggatgatgatgaagatgatgatgatgatgattttgatgatgaggaagctgaagaaaaagcgccagtgaagaaaggacaagaatccttcaagaaacaggaaaaaactcctaaaacaccaaaaggacctagttctgtagaagacattaaagcaaaaatgcaagcaagtatagaaaaaggtggttctcttcccaaagtggaagccaaattcatcaattatgtgaagaattgcttccggatgactgaccaagaggctattcaagatctctggcagtggaggaagtctctttaa

SEQ ID NO: 83 - DNA, 1185 bp, Homo sapiens
  source 1..1185: mol_type = genomic DNA
atgtcgggcgaggacgagcaacaggagcaaactatcgctgaggacctggtcgtgaccaagtataagatggggggcgacatcgccaacagggtacttcggtccttggtggaagcatctagctcaggtgtgtcggtactgagcctgtgtgagaaaggtgatgccatgattatggaagaaacagggaaaatcttcaagaaagaaaaggaaatgaagaaaggtattgcttttcccaccagcatttcggtaaataactgtgtatgtcacttctcccctttgaagagcgaccaggattatattctcaaggaaggtgacttggtaaaaattgaccttggggtccatgtggatggcttcatcgctaatgtagctcacacttttgtggttgatgtagctcaggggacccaagtaacagggaggaaagcagatgttattaaggcagctcacctttgtgctgaagctgccctacgcctggtcaaacctggaaatcagaacacacaagtgacagaagcctggaacaaagttgcccactcatttaactgcacgccaatagaaggtatgctgtcacaccagttgaagcagcatgtcatcgatggagaaaaaaccattatccagaatcccacagaccagcagaagaaggaccatgaaaaagctgaatttgaggtacatgaagtatatgctgtggatgttctcgtcagctcaggagagggcaaggccaaggatgcaggacagagaaccactatttacaaacgagacccctctaaacagtatggactgaaaatgaaaacttcacgtgccttcttcagtgaggtggaaaggcgttttgatgccatgccgtttactttaagagcatttgaagatgagaagaaggctcggatgggtgtggtggagtgcgccaaacatgaactgctgcaaccatttaatgttctctatgagaaggagggtgaatttgttgcccagtttaaatttacagttctgctcatgcccaatggccccatgcggataaccagtggtcccttcgagcctgacctctacaagtctgagatggaggtccaggatgcagagctaaaggccctcctccagagttctgcaagtcgaaaaacccagaaaaagaaaaaaaagaaggcctccaagactgcagagaatgccaccagtggggaaacattagaagaaaatgaagctggggactga

SEQ ID NO: 84 - DNA, 465 bp, Homo sapiens
  source 1..465: mol_type = genomic DNA
atggcgcagtctattaacatcacggagctgaatctgccgcagctagaaatgctcaagaaccagctggaccaggaagtggagttcttgtccacgtccattgctcagctcaaagtggtacagaccaagtatgtggaagccaaggactgtctgaacgtgctgaacaagagcaacgaggggaaagaattactcgtcccactgacgagttctatgtatgtccctgggaagctgcatgatgtggaacacgtgctcatcgatgtgggaactgggtactatgtagagaagacagctgaggatgccaaggacttcttcaagaggaagatagattttctaaccaagcagatggagaaaatccaaccagctcttcaggagaagcacgccatgaaacaggccgtcatggaaatgatgagtcagaagattcagcagctcacagccctgggggcagctcaggctactgctaaggcctga

SEQ ID NO: 85 - DNA, 330 bp, Homo sapiens
  source 1..330: mol_type = genomic DNA
atggcgcagtctattaacatcacggagctgaatctgccgcagctagaaatgctcaagaaccagctggaccagatgtatgtccctgggaagctgcatgatgtggaacacgtgctcatcgatgtgggaactgggtactatgtagagaagacagctgaggatgccaaggacttcttcaagaggaagatagattttctaaccaagcagatggagaaaatccaaccagctcttcaggagaagcacgccatgaaacaggccgtcatggaaatgatgagtcagaagattcagcagctcacagccctgggggcagctcaggctactgctaaggcctga

SEQ ID NO: 86 - DNA, 1320 bp, Homo sapiens
  source 1..1320: mol_type = genomic DNA
atgaatctgctgccgaatattgagagtccagtgactcggcaggagaagatggcgaccgtgtgggatgaggccgagcaagatggaattggggaggaggtgctcaagatgtccacggaggagatcatccagcgcacacggctgctggacagtgagatcaagatcatgaagagtgaagtgttgagagtcacccatgagctccaagccatgaaggacaagataaaagagaacagtgagaaaatcaaagtgaacaagaccctgccgtaccttgtctccaacgtcatcgagctcctggatgttgatcctaatgaccaagaggaggatggtgccaatattgacctggactcccagaggaagggcaagtgtgctgtgatcaaaacctctacacgacagacgtacttccttcctgtgattgggttggtggatgctgaaaagctaaagccaggagacctggtgggtgtgaacaaagactcctatctgatcctggagacgctgcccacagagtatgactcgcgggtgaaggccatggaggtagacgagaggcccacggagcaatacagtgacattgggggtttggacaagcagatccaggagctggtggaggccattgtcttgccaatgaaccacaaggagaagtttgagaacttggggatccaacctccaaaaggggtgctgatgtatgggcccccagggacggggaagaccctcctggcccgggcctgtgccgcacagactaaggccaccttcctaaagctggctggcccccagctggtgcagatgttcattggagatggtgccaagctagtccgggatgcctttgccctggccaaggagaaagcgccctctatcatcttcattgatgagttggatgccatcggcaccaagcgctttgacagtgagaaggctggggaccgggaggtgcagaggacaatgctggagcttctgaaccagctggatggcttccagcccaacacccaagttaaggtaattgcagccacaaacagggtggacatcctggaccccgccctcctccgctcgggccgccttgaccgcaagatagagttcccgatgcccaatgaggaggcccgggccagaatcatgcagatccactcccgaaagatgaatgtcagtcctgacgtgaactacgaggagctggcccgctgcacagatgacttcaatggggcccagtgcaaggctgtgtgtgtggaggcgggcatgatcgcactgcgcaggggtgccacggagctcacccacgaggactacatggaaggcatcctggaggtgcaggccaagaagaaagccaacctacaatactacgcctag

SEQ ID NO: 87 - DNA, 1395 bp, Homo sapiens
  source 1..1395: mol_type = genomic DNA
atggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaatcaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtatgcagatgttgaaggattctaa

SEQ ID NO: 88 - DNA, 1392 bp, Homo sapiens
  source 1..1392: mol_type = genomic DNA
atggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagggcctgcagttgccatcacccactgcaaccagccagctcccgctcgaatctgatgctgtggaatgcttaaattaccaacactataaaggaagtgactttgactgcgagttgaggctgttgattcatcagagtctagcaggaggaattattggggtcaaaggtgctaaaatcaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtattctggaaagtttttctaa

SEQ ID NO: 89 - DNA, 1320 bp, Homo sapiens
  source 1..1320: mol_type = genomic DNA
atggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagtaccaacactataaaggaagtgactttgactgcgagttgaggctgttgattcatcagagtctagcaggaggaattattggggtcaaaggtgctaaaatcaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtattctggaaagtttttctaa

SEQ ID NO: 90 - DNA, 1323 bp, Homo sapiens
  source 1..1323: mol_type = genomic DNA
atggaaactgaacagccagaagaaaccttccctaacactgaaaccaatggtgaatttggtaaacgccctgcagaagatatggaagaggaacaagcatttaaaagatctagaaacactgatgagatggttgaattacgcattctgcttcagagcaagaatgctggggcagtgattggaaaaggaggcaagaatattaaggctctccgtacagactacaatgccagtgtttcagtcccagacagcagtggccccgagcgcatattgagtatcagtgctgatattgaaacaattggagaaattctgaagaaaatcatccctaccttggaagagtaccaacactataaaggaagtgactttgactgcgagttgaggctgttgattcatcagagtctagcaggaggaattattggggtcaaaggtgctaaaatcaaagaacttcgagagaacactcaaaccaccatcaagcttttccaggaatgctgtcctcattccactgacagagttgttcttattggaggaaaacccgatagggttgtagagtgcataaagatcatccttgatcttatatctgagtctcccatcaaaggacgtgcacagccttatgatcccaatttttacgatgaaacctatgattatggtggttttacaatgatgtttgatgaccgtcgcggacgcccagtgggatttcccatgcggggaagaggtggttttgacagaatgcctcctggtcggggtgggcgtcccatgcctccatctagaagagattatgatgatatgagccctcgtcgaggaccacctccccctcctcccggacgaggcggccggggtggtagcagagctcggaatcttcctcttcctccaccaccaccacctagagggggagacctcatggcctatgacagaagagggagacctggagaccgttacgacggcatggttggtttcagtgctgatgaaacttgggactctgcaatagatacatggagcccatcagaatggcagatggcttatgaaccacagggtggctccggatatgattattcctatgcagggggtcgtggctcatatggtgatcttggtggacctattattactacacaagtaactattcccaaagatttggctggatctattattggcaaaggtggtcagcggattaaacaaatccgtcatgagtcgggagcttcgatcaaaattgatgagcctttagaaggatccgaagatcggatcattaccattacaggaacacaggaccagatacagaatgcacagtatttgctgcagaacagtgtgaagcagtatgcagatgttgaaggattctaa

SEQ ID NO: 91 - DNA, 90 bp, Mus musculus
  source 1..90: mol_type = genomic DNA
ggtgggaaaggaactgctcgtaggaagaagaaggtggttcacagaacggccacagcagacgataagaaactgcagttctccttaaagaag

SEQ ID NO: 92 - DNA, 90 bp, Mus musculus
  source 1..90: mol_type = genomic DNA
agagagaagaatgaaagtaacaagagcagctttgacaaaatgattgacgctatcaaagaaagcaagagcggcaagaagatcggagtgttc

SEQ ID NO: 93 - DNA, 90 bp, Mus musculus
  source 1..90: mol_type = genomic DNA
agcgctcctgaaggccaggcccaacaacgccggccctatcgcaggcgaaggttcccaccttactacatgcggagaccctatgcgcgtcga

SEQ ID NO: 94 - DNA, 90 bp, Mus musculus
  source 1..90: mol_type = genomic DNA
ggagagcaaggtagaccagtgagacagaatatgtatcggggttacagaccacgattccgaaggggccctcctcgccaaagacagcctaga

SEQ ID NO: 95 - DNA, 90 bp, Mus musculus
  source 1..90: mol_type = genomic DNA
cagccacctcaacgtcggtatcgccgaaacttcaattaccgacgcagacgcccagagaaccctaaaccacaagatggcaaagagacaaaa

SEQ ID NO: 96 - DNA, 90 bp, Mus musculus
  source 1..90: mol_type = genomic DNA
aatggaaaagacttaaaaccatcaacaccgagatcaaagggtcaagagtccttcaaaaaacaggaaaagactcctaaaacaccaaaagga

SEQ ID NO: 97 - DNA, 90 bp, Mus musculus
  source 1..90: mol_type = genomic DNA
aagggtcaagagtccttcaaaaaacaggaaaagactcctaaaacaccaaaaggacctagttctgtagaagacattaaggcaaaaatgcaa

SEQ ID NO: 98 - DNA, 90 bp, Mus musculus
  source 1..90: mol_type = genomic DNA
gtgtctgtgctgagcttgtgtgagaaaggtgatgccatgattatggaagagacagggaagatcttcaagaaggaaaaggagatgaagaaa

SEQ ID NO: 99 - DNA, 90 bp, Mus musculus
  source 1..90: mol_type = genomic DNA
gccaaggacttcttcaaaaggaagatagacttcctcaccaaacagatggagaaaatccagccagcgctgcaggagaagcatgccatgaag

SEQ ID NO: 100 - DNA, 90 bp, Mus musculus
  source 1..90: mol_type = genomic DNA
atcaagatcatgaagagtgaagtattgcgagtcacccatgaactccaagccatgaaagacaaaatcaaagagaacagtgagaaaatcaaa

SEQ ID NO: 101 - DNA, 90 bp, Mus musculus
  source 1..90: mol_type = genomic DNA
atgtttgatgaccgccgaggacgacctgtgggattccccatgaggggaagaggtggttttgacagaatgcctcctggtcggggtgggcgt

SEQ ID NO: 102 - DNA, 90 bp, Homo sapiens
  source 1..90: mol_type = genomic DNA
ggtgggaaaggaactgctcgcagaaagaagaaggtggttcatagaacagccacagcagatgacaaaaaacttcagttctccttaaagaag

SEQ ID NO: 103 - DNA, 90 bp, Homo sapiens
  source 1..90: mol_type = genomic DNA
cgagaaaagaatgaaagtaataagagtagctttgacaaaatgattgaagccattaaagaaagcaagaatggcaagaagattggagtgttc

SEQ ID NO: 104 - DNA, 90 bp, Homo sapiens
  source 1..90: mol_type = genomic DNA
agtgctcccgaaggccaggcccaacaacgccggccctaccgcaggcgaaggttcccaccttactacatgcggagaccctatgggcgtcga

SEQ ID NO: 105 - DNA, 90 bp, Homo sapiens
  source 1..90: mol_type = genomic DNA
ggagaacaaggtagaccagtgaggcagaatatgtatcggggatatagaccacgattccgcaggggccctcctcgccaaagacagcctaga

SEQ ID NO: 106 - DNA, 90 bp, Homo sapiens
  source 1..90: mol_type = genomic DNA
cagccacctcaacgtcggtaccgccgcaacttcaattaccgacgcagacgcccagaaaaccctaaaccacaagatggcaaagagacaaaa

SEQ ID NO: 107 - DNA, 93 bp, Homo sapiens
  source 1..93: mol_type = genomic DNA
aatggaaaagactcaaaaccatcatcaacaccaagatcaaaaggacaagaatccttcaagaaacaggaaaaaactcctaaaacaccaaaagga

SEQ ID NO: 108 - DNA, 90 bp, Homo sapiens
  source 1..90: mol_type = genomic DNA
aaaggacaagaatccttcaagaaacaggaaaaaactcctaaaacaccaaaaggacctagttctgtagaagacattaaagcaaaaatgcaa

SEQ ID NO: 109 - DNA, 90 bp, Homo sapiens
  source 1..90: mol_type = genomic DNA
gtgtcggtactgagcctgtgtgagaaaggtgatgccatgattatggaagaaacagggaaaatcttcaagaaagaaaaggaaatgaagaaa

SEQ ID NO: 110 - DNA, 90 bp, Homo sapiens
  source 1..90: mol_type = genomic DNA
gccaaggacttcttcaagaggaagatagattttctaaccaagcagatggagaaaatccaaccagctcttcaggagaagcacgccatgaaa

SEQ ID NO: 111 - DNA, 90 bp, Homo sapiens
  source 1..90: mol_type = genomic DNA
atcaagatcatgaagagtgaagtgttgagagtcacccatgagctccaagccatgaaggacaagataaaagagaacagtgagaaaatcaaa

SEQ ID NO: 112 - DNA, 90 bp, Homo sapiens
  source 1..90: mol_type = genomic DNA
atgtttgatgaccgtcgcggacgcccagtgggatttcccatgcggggaagaggtggttttgacagaatgcctcctggtcggggtgggcgt

SEQ ID NO: 113 - skipped

SEQ ID NO: 114 - protein, 7 aa, synthetic construct
  REGION 1..7: note = An artificially synthesized peptide sequence
PKKKRRV

SEQ ID NO: 115-117 - skipped (3 entries)

SEQ ID NO: 118 - protein, 16 aa, synthetic construct
  REGION 1..16: note = An artificially synthesized peptide sequence
KRPAATKKAGQAKKKK

SEQ ID NO: 119-120 - skipped (2 entries)

SEQ ID NO: 121 - protein, 27 aa, synthetic construct
  REGION 1..27: note = An artificially synthesized peptide sequence
FGNYNNQSSNFGPMKGGNFGGRSSGPY

SEQ ID NO: 122 - skipped

SEQ ID NO: 123 - protein, 27 aa, synthetic construct
  REGION 1..27: note = An artificially synthesized peptide sequence
GEGERPAQNEKRKEKNIKRGGNRFEPY

SEQ ID NO: 124 - protein, 43 aa, synthetic construct
  REGION 1..43: note = An artificially synthesized peptide sequence
VHSHKKKKIRTSPTFRRPKTLRLRRQPKYPRKSAPRRNKLDHY

SEQ ID NO: 125 - protein, 37 aa, synthetic construct
  REGION 1..37: note = An artificially synthesized peptide sequence
MSHRKFSAPRHGSLGFLPRKRSSRHRGKVKSFPKDDP

SEQ ID NO: 126 - protein, 50 aa, synthetic construct
  REGION 1..50: note = An artificially synthesized peptide sequence
EVTNDFVMLKGCVVGTKKRVLTLRKSLLVQTKRRALEKIDLKFIDTTSKF

SEQ ID NO: 127 - protein, 50 aa, synthetic construct
  REGION 1..50: note = An artificially synthesized peptide sequence
SLGQSASETEEDTVSVSKKEKNRKRRNRKKKKKPQRVRGVSSESSGDREK

SEQ ID NO: 128 - protein, 50 aa, synthetic construct
  REGION 1..50: note = An artificially synthesized peptide sequence
PTRYSVDIPLDKTVVNKDVFRDPALKRKARREAKVKFEERYKTGKNKWFF

SEQ ID NO: 129 - protein, 50 aa, synthetic construct
  REGION 1..50: note = An artificially synthesized peptide sequence
KMFKGKRGAQLAKDIARRSKTFNPGAGLPTDKKKGGPSPGDVEAIKNAIA

SEQ ID NO: 130 - protein, 4 aa, synthetic construct
  REGION 1..4: note = An artificially synthesized peptide sequence
KKEK